(19) United States
(12) Patent Application Publication
(10) Pub. No.: US 2021/0115430 A1
(43) Pub. Date: Apr. 22, 2021

(54) Immobilized Enzyme Complexes and Related Methods

(71) Applicants: Ronald WOOD, Belton, MO (US); The Curators of the University of Missouri, Columbia, MO (US)

(72) Inventors: Ronald Wood, Belton, MO (US); Hsin-Yeh HSIEH, Columbia, MO (US); Chung-Ho Lin, Columbia, MO (US); George Stewart, Columbia, MO (US); Mason Schellenberg, Columbia, MO (US); Kattesh Katti, Columbia, MO (US); Sagar Gupta, Columbia, MO (US); Shibu Jose, Columbia, MO (US)

(21) Appl. No.: 16/613,257

(22) PCT Filed: May 16, 2018
(86) PCT No.: PCT/US2018/033017

§ 371 Date: Nov. 13, 2019

Related U.S. Application Data
(60) Provisional application No. 62/506,864, filed on May 16, 2017.

(51) Int. Cl. C12N 11/089; C12N 11/14; C12M 1/40; C12P 19/14; A61K 9/70; C12P 19/02; C12P 7/64; A61K 38/48; A61K 47/42

(52) U.S. Cl. CPC C12N 11/089; C12N 11/14; C12M 21/18; C12P 19/14; A61K 47/42; C12P 19/02; C12P 7/649; A61K 38/48; A61K 9/7007

(57) ABSTRACT

Immobilized enzyme complexes (IEC) with enzymes that are non-covalently linked to matrices are provided along with methods for making the same. Methods of using the IEC for a wide variety of industrial enzymatic processes are also provided. Methods of converting cellulosic biomass and methods of effecting blood type conversions with the IEC are amongst the methods disclosed.

### BACKGROUND

High enzyme cost has been a bottleneck for commercial-scale success for industrial sectors that require enzymes in their manufacturing processes, such as the production of biofuels, specialty chemicals, pharmaceuticals and personal care products. For example, The 2007 U.S. Energy Independence and Security Act mandates that annual biofuel use nearly triple to 36 billion gallons per year (BGY) by 2022 with 21 BGY coming from advanced biofuels. Although cellulosic advanced biofuel production has been demonstrated on a pilot scale, the high enzyme cost associated with the saccharification process (the enzymatic hydrolysis of cellulose to sugars) has been a bottleneck for commercial-scale endeavors. Commercial-scale production will require transformational science that can significantly streamline the production process and significantly lower production costs.

In addition to the biofuel market, more than 100 different enzymatic biocatalytic processes have been implemented in pharmaceutical, chemical, agricultural, and food industries since 2000. The advantages of “green” biocatalytic processes over the traditional chemical processes include lower cost, higher product purity, and elimination of toxic chemicals and waste in the manufacturing process. The enzymatic process also significantly reduces the number of synthetic steps that would be required for conventional synthesis. Several classes of enzymes, including ketoreductases, transaminases, amine oxidases, mono-oxygenases and acyl transferases, have been used for a wide range of common chemical conversions in the manufacturing process of pharmaceuticals and specialty chemicals such as Telaprevir (Telavic, INCIVEK™), Sitagliptin (JANUVIA™), Simvastatin (Lipovas, ZOCOR™) Atazanavir (REYATAZ™), Esomeprazole (NEXIUM™), Atorvastatin (LIPITOR™), Montelukast (SINGULAIR™), Boceprevir (VICTRELIS™), and S-methoxyisopropylamine. In the food industry, enzymes such as amyloglucosidase and amylase glucose isomerases have been used to produce fructose syrups (sweeteners) from corn starch.

Reductions in enzyme costs can be achieved through improved immobilization of highly efficient enzymes. Immobilization of enzymes onto polymers is a growing field for enhancing biocatalytic activity and thermal and chemical stability of enzymes [1-4]. In addition, it allows the recovery and reuse of enzymes in biocatalytic processes. Cellulase has been immobilized by several physical and chemical methods, such as cross linking [5, 6], conjugation [2, 3], copolymerization [7], fiber ultrafiltration [8, 9], aqueous two-phase systems [10, 11] and modification of cellulase itself [12]. The immobilization of multi-enzyme complexes (artificial cellulosomes) via enzyme clustering could further improve the stability, storage properties, enzyme synergy, and catalytic efficacy in the saccharification process [1, 4]. Among the supporting platforms, nanoparticles are ideal supports for immobilization of cellulosomes, due to their minimum diffusional limitation, maximum specific surface area, and effective enzyme loading [1]. Recent studies showed that immobilization of enzymes enhanced biocatalytic activity (cellulose hydrolysis) via enzyme clustering by 2-7 folds in the enzymatic saccharification process [1, 4].

Enzyme-immobilization/clustering has been successfully demonstrated as a promising method to improve the efficiencies of sequential enzymatic reactions in enzymatic processes [5, 13]. Unfortunately, this strategy has not been economically viable for large-scale biomass processing because 1) enzymes cannot be efficiently recovered [14], 2) costs associated with enzyme purification is high, 3) enzyme specificity to the functionalized platform is low (and therefore requires enzyme purification), 4) supporting platforms cannot be regenerated or reused, and 5) linkers or conjugation agents used in the processes are often cost-prohibitive. Therefore, a novel approach is needed to make this process economically feasible for commercial-scale production. Recent advances in the development of the material synthesis, functionalization processes, conjugation chemistry and molecular engineering have made it possible to develop immobilized enzyme complexes to overcome the technical challenges described above.

### SUMMARY

Provided herein are immobilized enzyme complexes (IECs) comprising heat stable matrices that are covalently attached to biotin molecules or analogs thereof with linkers molecules and fusion proteins comprising enzyme domains and biotin binding domains (BBDs), wherein the biotin binding domains are non-covalently bound to biotin molecules or analogs thereof. In certain embodiments the heat stable matrix comprises carbon fiber, polystyrene, polylactic acid, polyurethane, silica, nylon, or polypropylene. In certain embodiments the heat stable matrix is selected from the group consisting of carbon fiber, polystyrene, polylactic acid, polyurethane, silica, nylon, and polypropylene. In certain embodiments the heat stable matrix is at least partially coated with a mixture of polyethylene glycol (PEG) and polyethyleneimine (PEI). In certain embodiments the linker molecule is attached to polyethyleneimine (PEI) molecules coating the matrix. In certain embodiments, one end or group of the linker molecule is attached to biotin or an analog thereof and another end or group of a linker molecule is attached to amine groups of polyethyleneimine (PEI) molecules coating the matrix. In certain embodiments, the linker molecule comprises an alkane group, an alkyl group, an amide, or combination thereof. In certain embodiments, biotin or an analog thereof is attached to a matrix by reacting a molecule comprising biotin or an analog thereof that is covalently linked to a C2 to C6 alkyl group that is covalently linked to a sulfo-N-hydroxysuccinimide (NHS) group with free amine groups of the matrix. In certain embodiments the heat stable matrix does not comprise a magnetic particle. In certain embodiments the biotin analog comprises desthiobiotin, 2′-iminobiotin, biotin sulfone, bisnorbiotin, tetranorbiotin, oxybiotin, any derivative thereof, or any derivative of biotin that can be bound by the BBD. In certain embodiments the enzyme domain is selected from the group consisting of a hydrolase, ketoreductase, transaminase, amine oxidase, mono-oxygenase, and an acyl transferase domain. In certain embodiments the enzyme domain is fused to the N-terminus of the BBD, to the C-terminus of the BBD, or to both the N-terminus and C-terminus of the BBD. In certain embodiments the enzyme domain is fused to either the N-terminus of the BBD or to the C-terminus of the BBD. In certain embodiments the enzyme domain is fused to the BBD with a peptide linker. In certain embodiments the enzyme domain is a glycoside hydrolase domain. In certain embodiments the glycoside hydrolase is an alpha-N-acetylgalactosaminidase, alpha-galactosidase, beta-glucosidase, a cellulase, an endoglucanase, or an exoglucanase. In certain embodiments, an amyloglucosidase and/or amylase glucose isomerase enzyme domain is used. In certain embodiments at least two fusion proteins are immobilized on the matrix. In certain embodiments the at least two fusion proteins comprise an enzyme domain that are each independently selected from the group consisting of a beta-glucosidase, an endoglucanase, and an exoglucanase. In certain embodiments, the beta-glucosidase, an endoglucanase, and an exoglucanase are ionic liquid tolerant, thermotolerant, or both. In certain embodiments at least one enzyme domain comprises a polypeptide having at least 70% sequence identity to a beta-glucosidase (SEQ ID NO: 2), an endoglucanase (SEQ ID NO: 3), an alpha N-acetylgalactosaminidase (SEQ ID NO: 4), an alpha-galactosidase of SEQ ID NO: 5-33, or 34. In certain embodiments the BBD comprises an avidin BBD, streptavidin BBD, tamavidin BBD, zebavidin BBD, bradavidin BBD, rhizavidin BBD, shwanavidin BBD, xenavidin BBD, a chimera thereof, or derivative thereof having one or more amino acid residue insertions, deletions, or substitutions. In certain embodiments the immobilized enzyme complex (IEC) or matrix is biocompatible. In certain embodiments the enzyme domain has proteolytic activity. In certain embodiments, the proteolytic activity is a collagenase activity. In certain embodiments the enzyme domain comprises a ketoreductase, transaminase, amine oxidase, mono-oxygenase, or acyl transferase domain. In certain embodiments the enzyme domain: (i) reduces RDX (hexahydro-1,3,5-trinitro-1,3,5-triazine); (ii) reduces 2,4,6-trinitrotoluene (TNT); (iii) reduces chromium 6+ to chromium 3+; or (iv) has 2,2′,3-trihydroxybiphenyl dioxygenase activity. In certain embodiments the IEC is contained in an enclosure that is permeable to a substrate and a product of the enzyme domain activity of (i) reducing RDX (hexahydro-1,3,5-trinitro-1,3,5-triazine); (ii) reducing 2,4,6-trinitrotoluene (TNT); (iii) reducing chromium 6+ to chromium 3+; or (iv) 2,2′,3-trihydroxybiphenyl dioxygenase, and comprises the enzyme domain of (i), (ii), (iii), or (iv), respectively. In certain embodiments, the enzyme domain comprises one or more sequences comprising enzyme domains that provide for atrazine degradation that are selected from the group consisting of SEQ ID NO:27-31, and 32. In certain embodiments, a combination of enzyme domains that provide for atrazine degradation that are selected from the group consisting of SEQ ID NO:27-31, and 32 are used. In certain embodiments the matrix is carbon fiber. In any of the aforementioned embodiments the IECs can be contained in bioreactor systems or in enclosures that are permeable to substrates and products of the enzyme domain-catalyzed conversions of the substrates. In any of the aforementioned embodiments the IECs can be adapted for application to subjects or objects in need thereof. In any of the aforementioned embodiments, the IEC can comprise a wound healing patch and the enzyme domain has proteolytic activity.

Also provided herein are bioreactors comprising any of the aforementioned immobilized enzyme complexes (IECs) configured for passage of liquids comprising substrates through the IECs. In certain embodiments the bioreactor apparatuses are configured for continuous flow of liquids through the IECs. In certain embodiments the bioreactors are configured for recirculation of liquids through the IECs.

Additionally provided herein are methods of enzymatic conversion of substrates to desired products comprising the steps of exposing the substrates to any of the aforementioned immobilized enzyme complexes as described herein under conditions where the substrates are converted to the desired products by exposure to the immobilized enzyme complexes. In certain embodiments the method further comprises the step of recovering the product. In certain embodiments the method further comprises: (i) removing the non-covalently bound fusion proteins from the matrix following conversion of substrate to a desired product; and (ii) binding fusion proteins to the matrix. In certain embodiments, the substrate is starch and an amyloglucosidase and/or an amylase glucose isomerase enzyme domain is used. In certain embodiments the substrate comprises cellulose and wherein the enzyme domains of at least one fusion proteins is selected from the group consisting of a beta-glucosidase, an endoglucanase, and an exoglucanase domain. In certain embodiments the substrate comprises whole blood or red blood cells and the enzyme domain of at least one fusion protein is selected from the group consisting of an alpha-N-acetylgalactosaminidase, alpha-galactosidase, or a combination thereof. In certain embodiments the enzyme domain: (i) reduces RDX (hexahydro-1,3,5 -trinitro-1,3,5 -triazine); (ii) reduces 2,4,6-trinitrotoluene (TNT); (iii) reduces chromium 6+ to chromium 3+; or (iv) has 2,2′,3-trihydroxybiphenyl dioxygenase activity. In certain embodiments the IEC is contained in an enclosure that is permeable to a substrate and product of the enzyme domain of (i) reducing RDX (hexahydro-1,3,5-trinitro-1,3,5-triazine); (ii) reducing 2,4,6-trinitrotoluene (TNT); (iii) reducing chromium 6+ to chromium 3+; or (iv) 2,2′,3-trihydroxybiphenyl dioxygenase and comprises the enzyme domain of (i), (ii), (iii), or (iv), respectively. In certain embodiments, the substrate is atrazine and the enzyme domain comprises one or more sequences comprising enzyme domains that provide for atrazine degradation that are selected from the group consisting of SEQ ID NO:27-31, and 32. In certain embodiments, a combination of enzyme domains that provide for atrazine degradation that are selected from the group consisting of SEQ ID NO:27-31, and 32 are used. In certain embodiments, the substrate is a wound, the IEC comprises a wound healing patch, and the enzyme domain has proteolytic activity.

Also provided herein are methods of making immobilized enzyme complexes, comprising: (a) covalently attaching biotin or analogs thereof dependent to heat stable matrices selected from the group consisting of a carbon fiber, polylactic acid, polyurethane, polystyrene, silica, nylon, and polypropylene by reacting said matrices with polyethylene glycol (PEG) and polyethyleneimine (PEI) at a ratio of 1 part PEG to 1.25 parts PEI to 1 part PEG to 3.5 parts PEI by weight and reacting the PEG/PEI-treated matrices with N-hydroxy-succinimide esters of biotin or biotin analogs to obtain a functionalized matrices; (b) removing any unreacted PEI, PEG, and esters of biotin or the biotin analogs from said functionalized matrices; and (c) non-covalently attaching at least one fusion protein comprising an enzyme domain and a biotin binding domain (BBD) to biotin or biotin analogs that are covalently attached to the functionalized matrices via linker molecules. In certain embodiments the methods further comprise: (i) removing the non-covalently bound fusion proteins from the matrix following conversion of substrate to a desired product by the attached fusion protein; and (ii) binding a fusion protein to the matrix. In certain embodiments the enzyme domain of at least one fusion protein is selected from the group consisting of an alpha-N-acetylgalactosaminidase, or alpha-galactosidase, or any combination thereof. In certain embodiments the enzyme is selected from the group consisting of a hydrolase, ketoreductase, transaminase, amine oxidase, mono-oxygenase, and an acyl transferase. In certain embodiments the ketoreductase, transaminase, amine oxidase, mono-oxygenase, or acyl transferase domain has a telaprevir precursor compound, sitagliptin precursor compound, or simvastatin precursor compound as a substrate. In certain embodiments the hydrolase is a glycoside hydrolase selected from the group consisting of an alpha-N-acetylgalactosaminidase, alpha-galactosidase, beta-glucosidase, a cellulase, an endoglucanase, and an exoglucanase. In certain embodiments the biotin analog comprises desthiobiotin, 2′-iminobiotin, biotin sulfone, bisnorbiotin, tetranorbiotin, oxybiotin, any derivative thereof, or any derivative of biotin that can be bound by the BBD. In certain embodiments the linker molecule comprises a C2 to C6 alkane group or a C2 to C6 alkyl group and an amide group. In certain embodiments, biotin or an analog thereof is attached to a matrix by reacting a molecule comprising biotin or an analog thereof that is covalently linked to a C2 to C6 alkyl group that is covalently linked to a sulfo-N-hydroxysuccinimide (NHS) group with free amine groups of the matrix. In certain embodiments the ratio of PEG to PEI is 1 part PEG to 1.5 parts PEI to 1 part PEG to 2.5 parts PEI by weight. In certain embodiments the enzyme domain of at least one fusion protein is selected from the group consisting of a beta-glucosidase, an endoglucanase, and an exoglucanase domain. In certain embodiments, the beta-glucosidase, an endoglucanase, and an exoglucanase are ionic liquid tolerant, thermos-tolerant, or both. In certain embodiments the enzyme domain has proteolytic activity. In certain embodiments, the proteolytic activity is a collagenase activity. In certain embodiments, an amyloglucosidase and/or amylase glucose isomerase enzyme domain is used. In certain embodiments the enzyme domain: (i) reduces RDX (hexahydro-1,3,5-trinitro-1,3,5-triazine); (ii) reduces 2,4,6-trinitrotoluene (TNT); (iii) reduces chromium 6+ to chromium 3+; or (iv) has 2,2′,3-trihydroxybiphenyl dioxygenase activity. In certain embodiments, the enzyme domain comprises one or more sequences comprising enzyme domains that provide for atrazine degradation that are selected from the group consisting of SEQ ID NO:27-31, and 32. In certain embodiments, a combination of enzyme domains that provide for atrazine degradation that are selected from the group consisting of SEQ ID NO:27-31, and 32 are used. In certain embodiments at least two fusion proteins are immobilized on the matrix. In certain embodiments the at least two fusion proteins comprise an enzyme domain that are each independently selected from the group consisting of a beta-glucosidase, an endoglucanase, and an exoglucanase. In certain embodiments at least one enzyme domain comprises a polypeptide having at least 70% sequence identity to a beta-glucosidase of SEQ ID NO: 2, an endoglucanase of SEQ ID NO: 3, alpha-N-acetylgalactosaminidase of SEQ ID NO: 4, an alpha-galactosidase of SEQ ID NO: 5, or an enzyme domain of SEQ ID NO:6-33, or 34. In certain embodiments the BBD comprises an avidin BBD, streptavidin BBD, tamavidin BBD, zebavidin BBD, bradavidin BBD, rhizavidin BBD, shwanavidin BBD, xenavidin BBD, a chimera thereof, or derivative thereof having one or more amino acid residue insertions, deletions, or substitutions.

Also provided herein are immobilized enzyme complexes made by any of the aforementioned methods described herein. In certain embodiments, the IEC comprises a wound healing patch and the enzyme domain enzyme domain has proteolytic activity.

Also provided herein are bioreactors, comprising: one or more immobilized fusion proteins bound to functionalized, biotinylated carbon fiber matrices to form a heat stable regenerative platform for genetically fused, engineered recombinant enzymes. In certain embodiments the bioreactor further comprises an enzyme comprising at least a portion of streptavidin or an analog. In certain embodiments the biotinylated matrices are propylene or an analog thereof. In certain embodiments the engineered recombinant enzymes are configured having one or more gene expression vector constructions cloned in a Biotin Binding Domain (BBD)-encoding open reading frame (ORF) built-in protein expression vector (pETstra) regulated by a T7 expression system. In certain embodiments the engineered recombinant enzymes are configured as streptavidin fused enzymes, antigens, antibodies, or peptides, and that are expressed by a protein expression system and attached onto a functionalized surface. In certain embodiments the functionalized surface is a biocompatible scaffold. In certain embodiments the bioreactor is configured as a continuous flow, multi-enzyme reactor system. In certain embodiments the bioreactor device is configured to form one or more therapeutic agents.

Additionally provided herein are methods of using a bioreactor, comprising the steps for methods of regeneration for recirculation of a liquid through an Immobilized Enzyme Complex. In certain embodiments the method further comprises steps for: recovering a desired product enzymatically converted from a substrate, exposing the substrate to the IEC under conditions, removing one or more non-covalently bound fusion proteins from a matrix following conversion of the substrate to the desired product, and binding fusion proteins to the matrix. In certain embodiments the method further comprises one or more steps for configuring a biofilter to maximize the surface area exposed to the immobilized enzyme complex wherein the substrate is converted to the desired product.

Also provided herein are continuous flow, multi-enzyme bioreactor systems, comprising: one or more engineered recombinant enzymes, genetically fused with streptavidin or another BBD, specific to a regenerated biofilter system having one or more functionalized platforms including a coating selected from a group consisting of carbon, agarose, polystyrene, polypropylene, polyurethane, silica, and nylon.

Also provided herein are Biofilter Systems, comprising: enzyme expression systems having one or more BBD or streptavidin-fused enzymes immobilized to at least one biotinylated meshed supporting media that are rapidly regenerated to form functionalized polymer platforms, wherein the biofilter is optionally immobilized with ionic liquid tolerant cellulases. In certain embodiments the Biofilter System immobilized with ionic liquid tolerant cellulases further comprises soluble cellulose extracted from biomass feedstock and an ionic liquid pretreatment process hydrolyzed by one or more thermophilic recombinant enzymes attached to a BBD. In certain embodiments the one or more thermophilic recombinant enzymes are selected from the group consisting of endoglucanases, exoglucanases, β-glucosidases from Trichoderma reesei, β-glucosidases from Aspergillus spp., thermophilic endoglucanase, Cel5A_Tma from Thermotoga maritima, β-1,4-endoglucanase (Cel5A) from Thermoanaerobacter tengcongensis MB4, endoglucanase and 1,4-beta-cellobiosidase from Paenibacillus spp. In certain embodiments the Biofilter System immobilized with ionic liquid tolerant cellulases is further configured to simultaneously convert free fatty acids and triglyceride into biodiesel, having an enzyme expression system immobilized with one or more lipases to facilitate enzymatic transesterification. In certain embodiments the Biofilter System immobilized with ionic liquid tolerant cellulases further comprises a biotinylated meshed supporting media and a filter to hydrolyze a soluble cellulose extracted from a biomass feedstock. In certain embodiments a multi-enzyme system is immobilized with one or more lipases to facilitate enzymatic transesterification process to simultaneously convert free fatty acids and triglyceride into biodiesel, and wherein the lipases are selected from a group consisting of Rhizopus oryzae and Candida rugosa. In certain embodiments, the lipases are selected from a group consisting of SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26.

### BRIEF DESCRIPTION OF THE DRAWINGS/FIGURES

FIG. 1. Expression vector design.

FIG. 2. Immobilized Enzyme Complex (IEC) utilization diagram for biomass conversion.

FIG. 3. Enzymatic activities for the engineered endo-cellulases fused with streptavidin (C- and N-terminal streptavidin fusion designated as “NT” or “CT” in the figure).

FIG. 4. The production of the major sugars and intermediates during catalytic depolymerization of cellulose.

FIG. 5. Derivatized PMP-Glucose [M+H⁺=511] analyzed by LC-MS at positive ion mode (A). The ion chromatogram of PMP-Glucose [M+H⁺=511] (B).

FIG. 6. Enzymatic activities for the engineered β-glucosidase fused with streptavidin.

FIG. 7. Western Blot analysis of recombinant endoglucanase (EGII) production. Lane 1: negative control; Lane2: EGII070914; Lane3: EGII071414; Lane4: EGII071614; LaneS: EGII072814; Lane6: EGII080114; Lane7: EGII080514@30° C.; Lane8: EGII080514@37° C.; Lane9: Protein molecular weight standards. Blue arrows indicated the band represented 55 kDa linker-fused EGII in each lane.

FIG. 8. The enzymatic activity bound to different matrix material: polystyrene A, B, C, D, single wall carbon nanomaterial (SWCNT 0.7-1.3 nm), silica beads, multiwall carbon nanomaterial (MWCNT-F, 0.5-10 μm), multiwall carbon nanomaterial (MWCNT-G, 5-9 μm), multiwall carbon nanomaterial (MWCNT-H, 2.5-20 μm), and agarose. Among these matrices, carbon material showed the best capacity for this recombinant enzyme.

FIG. 9. The concentrations of biotin on the surface of each matrix. Single wall carbon nanomaterial (SWCNT 0.7-1.3 nm), multiwall carbon nanomaterial (MWCNT-F, 0.5-10 μm), multiwall carbon nanomaterial (MWCNT-G, 5-9 μm), multiwall carbon nanomaterial (MWCNT-H, 2.5-20 μm).

FIG. 10. The cellulases immobilized on functionalized agarose (conjugated, solid circles) have shown higher thermal stability for production of sugars.

FIG. 11. The cellulases immobilized on functionalized agarose (immobilized, open bar) have shown longer shelf-life (8 days) as compared to free cellulases (solid bar 4 days and 8 days) for production of sugars.

FIG. 12. The shelf-life of β-glucosidase immobilized on the multiwall carbon nanomaterial (immobilized, black) have shown the activity until 8 days but free glucosidase (stripe-patterned) showed none beyond 4 days.

FIG. 13. The cellulases immobilized on functionalized silica (conjugated, diamonds) have shown enhanced enzymatic stabilities as compared to free cellulases (free, squares) for production of sugars: glucose (A) and cellobiose (B).

FIG. 14. The cellulases immobilized on functionalized agarose (immobilized, close square) have shown enhanced enzymatic stabilities as compared to free cellulases (free, open circle) for production of sugars: cellobiose (A) and cellotriose (B).

FIG. 15. The immobilization of multiple-enzymes (endoglucanase EGII and β-glucosidases βGL1) on the functionalized matrices has shown higher production of total sugars and glucose than either single class of enzyme alone. Cellulose is shown as upper section of bar and glucose is shown as lower section of bar.

FIG. 16. Effects of regeneration cycles on the proportion of enzyme immobilized on biotinylated agarose (black) to unbound enzyme (white). 86.17% of the recombinant enzyme could be immobilized to the agarose material after the first round of regeneration. The activity of enzyme bound was maintained above 72% in the rounds 2-4, and then it decreased below 50% in the 6th regeneration. The regeneration experiment has been stopped due to agarose degraded after the 6th heat de-attachment.

FIG. 17. Effects of regeneration cycles on the proportion of enzyme immobilized on biotinylated carbon material (black) to unbound enzyme (white). The highest enzyme bound to carbon particles was 91.5% in the 2nd round. The activity of enzyme bound was maintained above 50% up to six rounds, similar to the results with agarose beads. The carbon matrix exhibits good thermal stability characteristics.

FIG. 18. Reusability of Enzymes (The stability assay of β-Glucosidase immobilized SWCNT)—62.5 mg of β-glucosidase immobilized SWCNT was utilized in the enzymatic activity assay with 10 mM of pNPG substrate, incubated at 50° C. for 30 min. The measurement of OD₅₄₀ is in the function of the pNP production and a standard curve of pNP concentration at OD₅₄₀ was applied for the calculation of enzymatic activity (U mL⁻¹ min⁻¹). The β-glucosidase immobilized SWCNT was recovered at the end of incubations, washed with PBS, and reused in the next run of the same assay. The enzymatic activity assay was repeated 4 times with the recovered batch of samples in duplicate.

FIG. 19. pNP production (μmole) as the function of recombinant βGLI protein bound on carbon fiber (gm) functionalized by various ratios of PEG:PEI. The enzymatic reaction is in 10 mM pNPG substrate at 50° C. for 15 min. The test was to find the best ratio of PEG:PEI for functionalization that would provide the maximum of enzyme protein attachment. pNP production is the indicator for the amount of protein attached.

FIG. 20. Assay for activity of immobilized AagA protein—Enzymatic activity as a function of protein (mg) from crude extract of streptavidin-fused AagA protein expressing culture. A serial dilution of crude extract was made for protein samples in the enzymatic activity assay with pNP-NAG substrate in either 1 mM or 2.5 mM. The reaction was incubated at 37° C. for 15 min.

FIG. 21. Magnetic biocatalyst—Enzymatic activity as a function of βGLI immobilized carbon-iron particle—A serial of dilutions of βGLI immobilized carbon-iron particle and non-enzyme carbon-iron particle were made for the samples in duplicate for the enzymatic activity assay with 10 mM of pNPG substrate. The reactions were incubated at 50° C. for 15 min. The measurement of OD₅₄₀ is in the function of the pNP production and a standard curve of pNP concentration at OD₅₄₀ was applied for the calculation of enzymatic activity (U mL⁻¹ min⁻¹). The results showed that carbon-iron particle can be functionalized for linker-fused enzyme immobilization as other platform materials we tested with the advantage that it can be retrieved by magnetic power from the reactions.

FIG. 22. Production of sugars was increased by about ten-fold when β-glucosidase (βGLI) was immobilized on carbon fiber platforms as compared to free enzyme.

### DETAILED DESCRIPTION

Immobilized enzyme complexes (IECs) comprising fusion proteins with enzyme domains that are non-covalently attached to various matrices, methods of making the IECs, and methods of using the IECs are provided herein. Such IECs are suitable for a wide range of industrial processes including, but not limited to, biomass conversions, food product production, pharmaceutical production, blood type conversions, degradation of pollutants, and the like. Advantages of IECs provided herein can include, but are not limited to, improved enzyme stability in comparison to non-immobilized enzymes, efficient and/or cost effective purification of enzymes and manufacture of the IECs, and efficient and/or cost effective regeneration of IECs with new and/or different enzyme(s).

Matrices suitable for use in the IECs include, but not limited to, matrices that are heat stable. As used herein, the phrase “heat stable”, when used in reference to a matrix, refers to a matrix that is covalently attached to a biotin molecule or analog thereof that retains its ability to non- covalently bind the fusion protein comprising the enzyme domain following exposure to water, an aqueous liquid, or gaseous water at a temperature of at least 80° C. In certain embodiments, the matrices provided herein are heat stable at a temperature of at least 90° C. or 95° C. In certain embodiments, the matrices provided herein are heat stable at a temperature of 90° C. or 95° C. to 100° C., 110° C., 122° C., 130° C., or more. Non-limiting examples of heat stable matrices that can be used include, but are not limited to, carbon, carbon fibers (e.g. single wall carbon nanotubes (SWCNT), multi-wall carbon nanotubes (MWCNT), polystyrene, polylactic acid, polyurethane, silica, nylon, or polypropylene. In certain embodiments, the carbon matrices will be heat stable at temperatures of 80° C. to 100° C. or less than 104° C. In certain embodiments, the carbon fiber, polypropylene, or polyurethane matrices will be heat stable at temperatures of 80° C. to 100° C., 110° C., 122° C., 130° C., or more. In certain embodiments, the carbon fiber, polypropylene, or polyurethane matrices will be heat stable at temperatures of 80° C. to 100° C., 110° C., 122° C., 130° C., or more at elevated pressure, such as is achieved in an autoclave (e.g., 100 kPa (14.5 psi) or more. In certain embodiments, such heat stable matrices can provide for IECs that can be used at temperatures of 80° C. in conjunction with heat stable enzymes (e.g. engineered enzymes and/or enzymes obtained from hyper-thermophilic organisms). In certain embodiments, such heat stable matrices can provide for IECs that can be regenerated by removal of fusion proteins comprising spent enzyme domains by autoclaving and/or passage of water, aqueous solutions, or non-aqueous liquids at a temperature that will disrupt the non-covalent attachment of a fusion protein(s) comprising the spent enzyme domain followed by re-attachment of newly synthesized or other active fusion protein(s). IECs that are regenerable and methods of regenerating IECs are thus provided herein. As used herein, the term “spent enzyme domain” refer to an enzyme domain that has lost at least 10%, 20%, or 50% of its original enzymatic activity. Fusion proteins comprising spent enzyme domains can arise following conversion of substrate to a desired product by the fusion protein that is non-covalently attached to the matrix. In certain embodiments, removal of fusion proteins comprising spent enzyme domains from the heat stable matrix is effected by passage of water, an aqueous solution, or a non-aqueous liquid at a temperature of at least about 90° C. or 95° C. to 100° C. or more. In certain embodiments, removal of fusion proteins comprising spent enzyme domains can be effected with any of the aforementioned liquids or temperatures in conjunction with a denaturant that disrupts the non-covalent linkage of the fusion protein with the heat stable matrix. Examples of such denaturants include, but are not limited to, urea, thiourea, guanidine, sodium dodecyl sulfate, formamide, and the like.

Another component of the IECs provided herein are biotin molecules or analogs thereof that are attached to the heat stable matrices with linker molecules. Biotin analogues used in the IECs can include, but are not limited to, desthiobiotin, 2′-iminobiotin, biotin sulfone, bisnorbiotin, tetranorbiotin, oxybiotin, any derivative thereof, or any derivative of biotin that can be bound by a biotin binding domain (BBD). In certain embodiments, the biotin analog can exhibit reduced binding affinity (e.g., an increased disassociation constant or K_{d}) for the particular BBD of the fusion protein that is non-covalently bound to the biotin analog and the matrix. Non-limiting examples of biotin analogs with reduced binding affinity for a streptavidin BBD include, but are not limited to, desthiobiotin. Biotin or biotin analogs are covalently attached to the matrices via linker molecules. In certain embodiments, covalent attachment of the biotin or biotin analog is effected by an amide bond between a polyethyleneimine (PEI) polymer on the surface of the matrix and the linker molecule. Linker molecules attached to the surface of a matrix can comprise an alkane, an alkyl group, an amide, or combination thereof. In certain embodiments, and the alkane can comprise one or more of a C2 to C6 alkane(s). In certain embodiments, and the alkyl group can comprise one or more of a C2 to C6 alkyl group. In certain embodiments, two or more C2 to C6 alkanes or C2 to C6 alkyl groups are joined via one or more amide bonds in the linker molecule. In certain embodiments, covalent attachment of the biotin or biotin analog is effected by the reaction of an amine group of a polyethyleneimine (PEI) polymer on the surface of the matrix and a sulfo-NHS group of a linker molecule that is covalently linked to biotin. Linker molecules attached to the surface of a matrix can comprise an alkyl spacer, an Sulfo-NHS group that has reacted with an amine group of the matrix, or combination thereof Examples of biotin derivatives that further comprise linker molecule precursors include, but are not limited to, various biotin-N-hydroxysuccinimide esters. Commercially available biotin-N-hydroxysuccinimide esters that can be used include the Sulfo-NHS-Biotin, Sulfo-NHS-LC Biotin, and Sulfo-NHS-LC-LC Biotin products (Thermo, Carlsbad, Calif., USA). Biotin-N-hydroxysuccinimide esters can be reacted with matrices that have free amine groups to covalently link the biotin and linker molecule to the matrix via an amide bond to provide a functionalized matrix. As used herein, a “functionalized matrix” is a matrix having a biotin or biotin analog covalently attached thereto with a linker molecule. Such functionalized matrices include, but are not limited to, matrices where the biotin or biotin analog covalently attached thereto with an amide bond to the linker molecule that is attached to the biotin or biotin analog. Matrices with free amine groups can be prepared by a variety of methods. In certain embodiments, the matrix can be reacted with a mixture of polyethylene glycol (PEG) and polyethyleneimine (PEI) to form a polymer coat with free amines provided by the PEI. PEG and PEI can be coated on the matrix surface by mixing with water and baking onto the surface of the matrix. Coating of SWCNT with a 10 wt % solution of poly(ethyleneimine) (PEI, average molecular weight ˜25 kDa) and poly(ethylene glycol) (PEG, average molecular weight ˜10 kDa) in equal 1:1 ratios has been described by Star et al. (Nano Lett., Vol. 3, No. 4, 2003). In certain embodiments provided herein, reduced PEG:PEI ratios (i.e., less PEG than PEI) are used. In certain embodiments, PEG:PEI ratios of 1 part PEG to 1.25 parts PEI to 1 part PEG to 3.5 parts PEI by weight, 1 part PEG to 1.5 parts PEI to 1 part PEG to 2.5 parts PEI by weight, or 1 part PEG to 1.8 parts PEI to 1 part PEG to 2.2 parts PEI by weight are used to coat the matrix. In certain embodiments, about 1 part PEG to about 2 parts PEI by weight are used to coat the matrix. IECs with increased amounts of immobilized enzyme domains can be obtained by using such reduced PEG:PEI ratios. Removal of unreacted PEG and PEI can be effected by rinsing the treated matrices with water, aqueous solutions, and the like. PEG/PEI treated matrices that have been rinsed are in certain embodiments subjected to a subsequent heating or drying step. Biotin-N-hydroxysuccinimide esters comprising linker molecules can be reacted with PEG/PEI treated, rinsed, and dried matrices to provide functionalized matrix. Non-covalent attachment of the fusion protein to the functionalized matrix can be effected by contacting the functionalized matrix with the fusion protein.

Fusion proteins comprising enzyme domains and BBDs can be constructed by recombinant DNA techniques wherein nucleic acids encoding those domains are joined such that a single open reading frame encoding both domains is created. As used herein, the phrase “enzyme domains” refers to a portion of an enzyme that can convert any substrate of the enzyme to a reaction product. It is thus recognized that an enzyme domain can in certain embodiments comprise a less than complete part of an enzyme so long as it retains at least some enzymatic activity. In certain embodiments, the enzyme domain can thus comprise an N-terminal deletion, a C-terminal deletion, an internal deletion, or any combination of such deletions of one, two, three, or more amino acid residues of a protein containing the enzyme domain. In certain embodiments, the enzyme domain can comprise one, two, three, or more amino acid residue substitutions. In certain, embodiments the enzyme domain can comprise one, two, three, or more amino acid residue substitutions in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In certain embodiments, the enzyme domain can comprise the sequences of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 having one, two, three, or more and/or one or more of an N-terminal deletion, a C-terminal deletion, an internal deletion, or any combination of such deletions of one, two, three, or more amino acid residues. In certain embodiments, the enzyme domain can comprise a protein having at least 70%, 80%, 85%, 90%, 95%, 97%, 98%, or 100% sequence identity to SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In other embodiments, a nucleic acid sequence encoding the full length or mature enzyme sequence that contains the enzyme domain can be used. Nucleic acids encoding the BBD can be fused in frame to nucleic acids encoding the enzyme domain to produce either an N-terminal or C-terminal fusion protein suitable for use in the IECs provided herein.

The Biotin Binding Domain (BBD) used in the fusion proteins can be obtained from a wide variety of proteins or can be engineered. As used herein, the phrase “Biotin Binding Domain” or “BBD” refers to a portion of a protein that can bind to biotin or an analogue thereof. It is thus recognized that a BBD can, in certain embodiments, comprise a less than complete part of an protein so long as it retains at least some biotin or biotin analogue binding activity. In certain embodiments, the BBD or protein comprising the BBD will have a dissociation constant (K_{d}) for biotin or biotin analogue of at least about 7×10⁻⁵ M, 1×10⁻⁶ M, 1×10⁻⁷ M, 1×10⁻⁸ M, or 1×10⁻⁹ M to about 1×10⁻¹² M, 1×10⁻¹³ M, 1×10⁻¹⁴ M, or 1×10⁻¹⁵ M. In certain embodiments, the enzyme domain can thus comprise an N-terminal deletion, a C-terminal deletion, an internal deletion, or any combination of such deletions of one, two, three, or more amino acid residues of a protein containing the BBD. In other embodiments, a nucleic acid sequence encoding the full length or mature protein sequence that contains the BBD can be used. In certain embodiments, the full length or mature protein that is used for the BBD that is used can comprise an avidin BBD, streptavidin BBD, tamavidin BBD, zebavidin BBD, bradavidin BBD, rhizavidin BBD, shwanavidin BBD, xenavidin BBD, a chimera thereof, or derivative thereof having one or more amino acid residue insertions, deletions, or substitutions. As used herein in this context, the term “chimera” refers to a protein comprising a BBD that has amino acid sequences of at least two proteins that contain a BBD. In certain embodiments, the fusion protein comprising the BBD will be able to form a homotetramer that binds biotin or an analogue thereof In certain embodiments, the protein comprising the BBD can bind biotin or an analogue thereof as a monomer. Amino acid substitutions in streptavidin that provide for monomeric proteins that bind biotin with a K_{d} of about 1×10⁻⁸ M include, but are not limited to, T90A and D128A amino acid substitutions (Qureshi M H, Wong S L. Protein Expr. Purif. 25(3):409-15, 2002). Amino acid substitutions in streptavidin that provide for proteins that bind biotin at a K_{d} of less than 1×10⁻¹⁰ M include, but are not limited to, W79A, W120A, and W120F (Chilkoti A, et al. PNAS-USA 1995;92(5):1754-1758), and N23A, S27D, and S45A (Howarth et al. Nature Methods. 2006;3(4):267-273). In certain embodiments, it is thus contemplated that proteins comprising streptavidin (SEQ ID NO: 1) or derivatives thereof having one, two, three, four, or more amino acid substitutions, deletions, insertions, or any combination thereof and that comprise a BBD can be used in the IEC. In certain embodiments, the protein comprising the BBD used in the IEC will have at least 70%, 80%, 85%, 90%, 95%, 97%, 98%, or 100% sequence identity to SEQ ID NO: 1.

Nucleic acids encoding the aforementioned fusion proteins can be operably linked to suitable promoters and other sequences including, but not limited to, 5′ and/or 3′ untranslated regions, sequences encoding secretion signal peptides, ribosome binding sites, termination sequences, polyadenylation sequences, and the like, incorporated into suitable transformation vectors, and introduced into suitable host cells that express the fusion protein. A host cell can be any prokaryotic (e.g., E. coli) or eukaryotic cell (e.g., insect cells, yeast, plant, or mammalian cells). The recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers, ribosome binding sites, transcriptional terminators, and other expression control elements (e.g., polyadenylation signals). Regulatory sequences include those which direct constitutive expression of a nucleotide sequence in many types of host cell and those which provide for inducible expression. Such operably linked sequences as described above are tailored for use in prokaryotic (e.g., E. coli) or eukaryotic cells (e.g., insect cells (using baculovirus expression vectors), yeast cells, plant cells, or mammalian cells). Examples of suitable inducible non-fusion E. coli expression vectors include, but are not limited to, pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector can rely on transcription from a T7 gn10-lac fusion promoter mediated by a co-expressed viral RNA polymerase (T7 gni). This viral polymerase can be supplied by host strains BL21(DE3) or HMS174(DE3) from a resident prophage harboring a T7 gnl gene under the transcriptional control of the lacUV 5 promoter. Examples of vectors for expression in yeast S. cerevisiae or P. pastoris include, but are not limited to, pYepSec1 (Baldari et al., (1987) EMBO J. 6:229-234), pMFa (Kurjan and Herskowitz, (1982) Cell 30:933-943), pJRY88 (Schultz et al., (1987) Gene 54:113-123), pYES2 (ThermoFischer, Carlsbad, Calif.), and pPicZ (ThermoFischer, Carlsbad, Calif.). For expression in Pichia, a methanol-inducible promoter is preferably used. In certain embodiments, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Luckow and Summers (1989) Virology 170:31-39). In certain embodiments, the fusion protein is expressed in mammalian cells using a mammalian expression vector. Mammalian expression vectors include, but are not limited to, pCDM8 (Seed (1987) Nature 329:840) and pMT2PC (Kaufman et al., (1987) EMBO J. 6:187-195). When used in mammalian cells, the expression vector's control functions are often provided by viral regulatory elements. For example, commonly used promoters are derived from polyomavirus, Adenovirus 2, cytomegalovirus and Simian Virus 40. Other suitable expression systems for both prokaryotic and eukaryotic cells are described in Sambrook et al. (Molecular Cloning: A Laboratory Manual, 4^{th} Ed., Cold Spring Harbor Press, 2012). Alteration of the nucleic acid sequence of the nucleic acid to be inserted into an expression vector so that the individual codons for each amino acid are those more commonly used in the target host cell (e.g., prokaryotic or eukaryotic host cell “codon optimization”) is also provided herein.

Vectors that provide for extracellular expression of the fusion proteins can also be used in certain embodiments. In such vectors, secretion signal sequences that provide for secretion of fusion proteins in the desired host cell are operably linked to the N-terminus of the fusion protein. Prokaryotic secretion signals that can be used include, but are not limited to, alkaline phosphatase signal peptides and the like. Mammalian secretion signals include, but are not limited to, a tPA signal peptide, a mammalian alkaline phosphatase signal peptide and the like. Yeast secretion signals include, but are not limited to, a yeast alpha mating type signal peptide, a yeast invertase signal peptide, or yeast alkaline phosphatase signal peptide and the like. Insect cell secretion signals include, but are not limited to, an egt signal peptide, a p67 signal peptide, or other signal peptides useful for expression of heterologous proteins as disclosed in U.S. Pat. No. 5,516,657.

Vector DNA encoding the fusion protein can be introduced into prokaryotic or eukaryotic cells via conventional transformation techniques. As used herein, the terms “transformation” includes any method whereby an exogenous nucleic acid is introduced into a cell. Transformation methods thus include, but are not limited to, calcium phosphate or calcium chloride co-precipitation, DEAE-dextran-mediated transfection, lipofection, particle mediated delivery, heat shock, electroporation, transfection or viral transduction. To obtain transformed cells, a gene that encodes a selectable marker is generally introduced into the host cells along with the gene of interest. For prokaryotic cells, selectable markers include, but are not limited to, genes that confer resistance to antibiotics, genes that confer the ability to grow in the absence of otherwise required nutrients, and the like. For eukaryotic cells, selectable markers that confer resistance to drugs including, but not limited to, G418, hygromycin, ZEOCIN™ and methotrexate and genes that confer the ability to grow in the absence of otherwise required nutrients, and the like can be used.

Fusion proteins can be obtained from the host cells by culturing the cells under conditions where the fusion protein is expressed and either lysing or otherwise disrupting the cells to release the intracellular fusion protein or by harvesting the fusion protein from the culture media when the host cells secrete the fusion protein. Conditions where the fusion protein is expressed include, but are not limited to, conditions where the expression of the fusion protein is induced (e.g., such as by induction of a promoter that is operably linked to a nucleic acid encoding the fusion protein). In certain embodiments, the IEC is made by contacting any of the aforementioned matrices with biotin or a biotin analogue covalently linked to a fusion protein obtained from a host cell or from the culture media in which the host cell was grown to permit non-covalent binding of the fusion protein to the matrix. Contacting conditions are adapted to permit the BBD of the fusion protein to bind to the biotin or biotin analogue that is covalently linked to the matrix. In certain embodiments, the IEC can be contacted with a crude or minimally purified lysate from the host cell or with host cell culture media or a concentrate thereof that comprises the fusion protein. In other embodiments, the cell lysate, cell culture media, or concentrate thereof containing the fusion protein can be subjected to one or more purification or enrichment steps. Examples of such purification or enrichment steps include, but are not limited to, at least partial removal of carbohydrates, lipids, glycoproteins, proteins of higher and/or lower molecular weight than the fusion protein, and the like, via size exclusion, high pressure liquid chromatography, ion exchange chromatography, affinity chromatography, and combinations thereof

In certain embodiments, IEC provided herein can be used in a bioreactor. Bioreactors include, but are not limited to, apparatuses that provide for contacting the IEC with substrates of the enzyme domains of the immobilized fusion proteins continuously, semi-continuously, in batch mode, in fed batch mode, or in any combination thereof. In certain embodiments, solutions containing enzyme domain substrates are passed through the bioreactor containing the IEC once, or are passed through the bioreactor containing the IEC at least two, three, or more times. In certain embodiments, passage of a solutions containing enzyme domain substrates through the IEC-containing bioreactor can be performed in a closed loop system such that the solution that originally contained the substrate is passed through the bioreactor at least two, three, or more times or until the substrate is depleted. In addition, the soluble cellulose extracted from biomass feedstock using an ionic liquid (IL) pretreatment process can be hydrolyzed by the immobilized multi- enzyme complex in the continuous-flow bio-filter system. In certain embodiments, depletion of the substrate from a solution can comprise reductions in the original substrate concentration of at least 50%, 75%, 85%, 90%, 95%, 98%, or 99%.

In certain embodiments, IEC provided herein can be contained in an enclosure that is permeable to a substrate and a product of the enzyme domain activity. In still other embodiments, the enclosure that is permeable to a substrate and a product of the enzyme domain activity can be incorporated into a bioreactor, including, but not limited to, any of the aforementioned bioreactors. In certain embodiments, enclosures used in this manner can comprise a membrane having a pore size with a molecular weight cutoff (MWCO) that will permit the substrate to enter the enclosure and allow the reaction product to leave the enclosure. As used herein, a “molecular weight cutoff” or “MWCO” of a membrane refers to the lowest molecular mass of a solute molecule that will be retained by the membrane by at least 90% (i.e., at least 90% of the solute molecule that was originally contained by the membrane is retained). Membranes used in such enclosures can be selected based on considerations including, but not limited to, the molecular weights of the substrate and product of the immobilized membrane domain, the presence of other elements in the solution that are desirable to exclude from the enclosure, desired diffusion rates for the substrate and product, and the like. In certain embodiments, the membrane has a MWCO of about 1, 2, or 5 kDa to about 8, 10, 20, 50, 100, 300, 500, or 1000 kDa. IEC enclosed in such membranes can be used in methods of degrading various pollutants. In certain embodiments, enzyme domains of an XplA-XplB cytochrome P450 from Rhodococcus spp., variants of, or other cytochrome P450s that degrade hexahydro-1,3,5-trinitro-1,3,5-triazine (RDX) can be used in an IEC to remove RDX. In certain embodiments, NADPH nitroreductase enzyme domains that recognize 2,4,6-trinitrotoluene (TNT), including PnrA from Pseudomonas putida, variants thereof, or other NADPH nitroreductase enzymes for degrading TNT can be used in an IEC to remove TNT. In certain embodiments, enzyme domains from enzymes that degrade TNT disclosed in Esteve-Núñez A, et al. Microbiology and Molecular Biology Reviews. 2001;65(3):335-352 can be used. In certain embodiments, dioxin dioxygenase enzyme domains including, but not limited to, dxnA1-A2/DbfB from Sphingomonas spp., variants thereof, or other dioxin dioxygenases can be used in an IEC for removing dioxin. In certain embodiments, chromate reductase enzyme domains including but not limited to ChrR chromate reductase enzyme domains from Pseudomonas putida, variants thereof, or other chromate reductase enzyme domains for reducing chromium 6+ to chromium 3+. In certain embodiments, the matrices used in the aforementioned IEC and related methods are carbon fiber matrices.

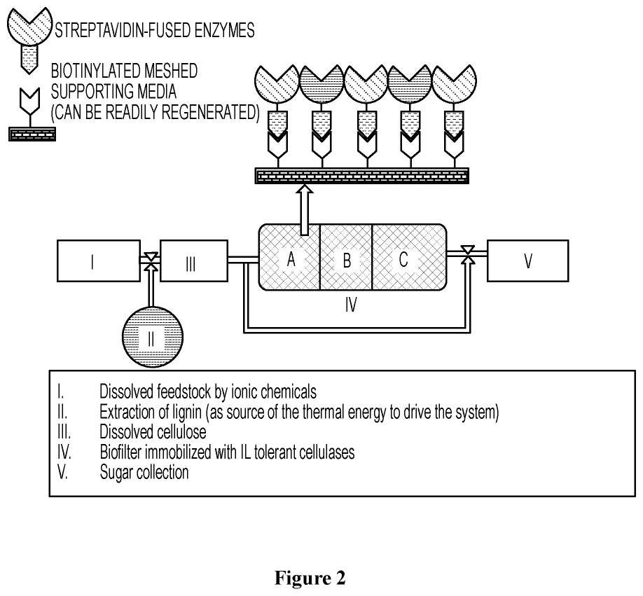

In certain embodiments, the immobilized enzyme complex (IEC) could be used to construct a multi-enzyme bioreactor or bio-filter system for production of cellulosic biofuel or any other useful product of a reaction catalyzed by the immobilized enzymes. Methods for using such bioreactors are also provided herein. A non-limiting example of how a multi-enzyme IEC could be used in cellulosic biofuel is shown in FIG. 2. In one embodiment, the IEC could be utilized to directly convert the soluble sugars (e.g., cellopentaose, cellotriose, and cellobiose) to glucose. In addition, the soluble cellulose extracted from biomass feedstock using an ionic liquid (IL) pretreatment process can be hydrolyzed by the immobilized multi-enzyme complex in the continuous-flow bio-filter system. In certain embodiments, thermo-tolerant recombinant enzyme domains, enzyme domains that that are tolerant to IL chemicals, or enzyme domains that are both thermo-tolerant and IL-tolerant can be used. In certain embodiments, the IL-tolerant enzyme domains can exhibit less than 50%, 40%, 30%, 20%, 10% or 5% reductions in enzymatic activity in comparison to an IL intolerant enzyme domain when exposed to the same concentration of the IL. In certain embodiments, the thermo-tolerant enzyme domains can exhibit less than 50%, 40%, 30%, 20%, 10% or 5% reductions in enzymatic activity in comparison to a thermo-sensitive enzyme domain (e.g., wild-type enzyme domain) when exposed to the same temperature. These enzymes can include, but are not limited to endoglucanases, exoglucanases, and β-glucosidases from Trichoderma reesei and Aspergillus spp., thermophilic endoglucanase, Cel5A_Tma and endo-1,4-□-xylanase A from Thermotoga maritama, β-1,4-endoglucanase (Cel5A) from Thermoanaerobacter tengcongensis MB4, endoglucanase and 1,4-□-cellobiosidase from Paenibacillus spp, and alpha-L-arabinofuranosidase A-like protein from Bifidobacterium thermophilum. In certain embodiments, 1-butyl-3-methylimidazolium chloride, 1-ethyl-3-methylimidazolium acetate and 1-allyl-3-methylimidazolium chloride can be used as pretreatment IL chemicals for the immobilized recombinant enzymes. In certain embodiments, the pretreatment IL chemicals are used with β-1,4-endoglucanase (Cel5A) of Thermoanaerobacter tengcongensis MB4 and Cel5A_Tma, a thermophilic endoglucanase from Thermotoga maritama, which are resistant to certain IL ionic liquids [16, 22]. In certain embodiments, the enzyme domain(s) can comprise the sequences of SEQ ID NO: 2, SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 34 having one, two, three, or more and/or one or more of a N-terminal deletion, a C-terminal deletion, an internal deletion, or any combination of such deletions of one, two, three, or more amino acid residues. In certain embodiments, the enzyme domain(s) can comprise a protein having at least 70%, 80%, 85%, 90%, 95%, 97%, 98%, or 100% sequence identity to SEQ ID NO: 2, SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 34.

In certain embodiments, β-1,4-endoglucanase (Cel5A) from Thermoanaerobacter tengcongensis MB4, which is also remarkably resistant in ionic liquids 1-butyl-3-methylimidazolium chloride and 1-allyl-3-methylimidazolium chloride, is used in the IEC or used in conjunction with those ionic liquids in the IEC. It has been shown that IL tolerance can be correlated to themostability and halotolerance. In certain embodiments, enzyme domains of several cellulases isolated from Aspergillus species have been shown to be halotolerant, have excellent tolerance to the ILs, and can be used in the IEC. In certain embodiments, lower IL concentrations (25-50% w/v) in water are used with the IEC. Such lower concentrations of ILs are not only effective for pretreating biomass, but also protect stability of the enzymes during the saccharification process. In certain embodiments, the immobilized cellulase stability could also be further improved by coating the immobilized cellulases with hydrophobic ILs such as butyltrimethylammonium bis(trifluoromethylsulfonyl)imide ([N1114][NTf2]). Hydrophobic ILs ([N1114][NTf2] have been used to enhance the stability of the immobilized cellulases in ILs by 4 times. This strategy has been successfully used for the saccharification of dissolved cellulose in 1-butyl-3-methylimidazolium chloride ([Bmim][C1]) (i.e. up to 50% hydrolysis in 24 h) at 50° C. and 1.5 w/v water content.

Also provided herein are IECs, bioreactors comprising the same, and related methods that can convert type A, B, or AB blood or blood cells to type O blood or blood cells. Conversion of A blood group antigens by the AagA gene product of Clostridium perfringens which comprises an alpha-N-acetylgalactosaminidase has been reported (Calcutt et al. FEMS Microbiology Letters. 214 (2002) 77-80). In certain embodiments, alpha-galactosidases which remove galactose residues, at the non-reducing end of carbohydrate precursor chain and convert B antigen into H antigen are used in the IEC. A combination of an alpha-N-acetylgalactosaminidase and an alpha-galactosidase can be used to convert A, B, or AB blood or blood cells to type O blood or blood cells. In certain embodiments, the enzyme domain used in the IEC can comprise an alpha-N-acetylgalactosaminidase, an alpha-N-acetylgalactosaminidase of SEQ ID NO: 4, or a variant thereof. In certain embodiments, the variant enzyme domain can comprise the sequences of SEQ ID NO: 4 having one, two, three, or more and/or one or more of a N-terminal deletion, a C-terminal deletion, an internal deletion, or any combination of such deletions of one, two, three, or more amino acid residues. In certain embodiments, the variant enzyme domain can comprise a protein having at least 70%, 80%, 85%, 90%, 95%, 97%, 98%, or 100% sequence identity to SEQ ID NO: 4. Alpha-galactosidases containing enzyme domains suitable for use in the IEC include, but are not limited to, those from coffee bean (Zhu et al., (1996) Arch Biochem Biophys. 15;327(2):324-9; SEQ ID NO: 5), pinto bean (Davis et al., (1997) Biochem Mol Biol Int., July;42(3):453-67;), and soybean (Davis et al. (1996) Biochem Mol Biol Int. June;39(3):471-85), and variants thereof In certain embodiments, the enzyme domain used in the IEC can comprise an alpha-galactosidase, an alpha-galactosidase of SEQ ID NO: 5 or a variant thereof In certain embodiments, the variant enzyme domain can comprise the sequences of SEQ ID NO: 5 having one, two, three, or more and/or one or more of a N-terminal deletion, a C-terminal deletion, an internal deletion, or any combination of such deletions of one, two, three, or more amino acid residues. In certain embodiments, the variant enzyme domain can comprise a protein having at least 70%, 80%, 85%, 90%, 95%, 97%, 98%, or 100% sequence identity to SEQ ID NO: 5. In certain embodiments, the enzyme domain used in the IEC can comprise SEQ ID NO: 6, SEQ ID NO: 7, SEQ ID NO: 8, SEQ ID NO: 9, SEQ ID NO: 10, or variants thereof. In certain embodiments, the variant enzyme domain can comprise the sequences of SEQ ID NO: 6, SEQ ID NO: 7, SEQ ID NO: 8, SEQ ID NO: 9, or SEQ ID NO: 10 having one, two, three, or more and/or one or more of a N-terminal deletion, a C-terminal deletion, an internal deletion, or any combination of such deletions of one, two, three, or more amino acid residues. In certain embodiments, the variant enzyme domain can comprise a protein having at least 70%, 80%, 85%, 90%, 95%, 97%, 98%, or 100% sequence identity to SEQ ID NO: 6, SEQ ID NO: 7, SEQ ID NO: 8, SEQ ID NO: 9, or SEQ ID NO: 10.

Other applications of the IEC systems provided herein include, but are not limited to: wound healing patches (e.g., proteolytic enzymes, such as papain or collagenase enzyme domains), fuel cells (enzyme based biological fuel cells), starch conversion to fructose (e.g., using amyloglucosidase and/or amylase glucose isomerase enzyme domains), drug delivery systems (e.g., antimicrobial proteins: lysozyme, etc.), flavor removal, stain eliminator (immobilized URINASE™ or protease enzyme domains), biosurfactants and detergents (enzyme domains of proteases, lipases as biosurfactants and detergents for industrial use, e.g. wetting, degreasing, soaking agents in tanning/food industry) and bio-filters (e.g., XplA-XplB cytochrome P450 from Rhodococcus spp. for removing RDX, PnrA from Pseudomonas putida for removing TNT, dioxin dioxygenase (dxnA1-A2/DbfB) from Sphingomonas spp. for removing dioxin, and ChrR chromate reductase from Pseudomonas putida for reducing chromium 6+ to chromium 3+). In certain embodiments, the lipase can comprise the sequences of SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26 having one, two, three, or more and/or one or more of a N-terminal deletion, a C-terminal deletion, an internal deletion, or any combination of such deletions of one, two, three, or more amino acid residues. In certain embodiments, the variant enzyme domain can comprise a protein having at least 70%, 80%, 85%, 90%, 95%, 97%, 98%, or 100% sequence identity to SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26. In certain embodiments, the enzyme domain comprises one or more sequences comprising enzyme domains that provide for atrazine degradation that are selected from the group consisting of SEQ ID NO:27-31, and 32. In certain embodiments, fragments of SEQ ID NO:27-31, and 32 that comprise the enzyme domains of those sequences that provide for atrazine-degrading activity are used. In certain embodiments, a combination of enzyme domains that provide for atrazine degradation that are selected from the group consisting of SEQ ID NO:27-31, and 32 are used. In certain embodiments, the enzyme domain can comprise the sequences of SEQ ID NO: 27, SEQ ID NO: 28, SEQ ID NO: 29, SEQ ID NO: 30, SEQ ID NO: 31, SEQ ID NO: 32, or SEQ ID NO: 33 having one, two, three, or more and/or one or more of a N-terminal deletion, a C-terminal deletion, an internal deletion, or any combination of such deletions of one, two, three, or more amino acid residues. In certain embodiments, the variant enzyme domain can comprise a protein having at least 70%, 80%, 85%, 90%, 95%, 97%, 98%, or 100% sequence identity to SEQ ID NO: 27, SEQ ID NO: 28, SEQ ID NO: 29, SEQ ID NO: 30, SEQ ID NO: 31, SEQ ID NO: 32, or SEQ ID NO: 33.

Immobilized enzyme complexes provided herein can also be adapted for application to a subject or object in need thereof. Such adaptions include, but are not limited to, use of biocompatible materials as the matrices of the IEC. In certain embodiments, biocompatible materials will not elicit an adverse reaction in the subject. Application methods include, but are not limited to, any parenteral administration (e.g., intravenous, intra-arterial, intramuscular, subcutaneous, intradermal, intraperitoneal, or intrathecal delivery) topical administration, oral administration, and mucosal administration (e.g., intranasal, inhalation, rectal, vaginal, buccal, or sublingual delivery). Subjects include, but are not limited to animals, humans, plants, and plant parts including leaves, seeds, flowers, and the like. Non-limiting examples of a subject in need include, but are not limited to, subjects suffering from infection to which an IEC comprising an antimicrobial protein or enzyme domain (e.g. lysozyme) is applied.

### EXAMPLES

### Example 1

### Manufacture of Biotinylated Matrices

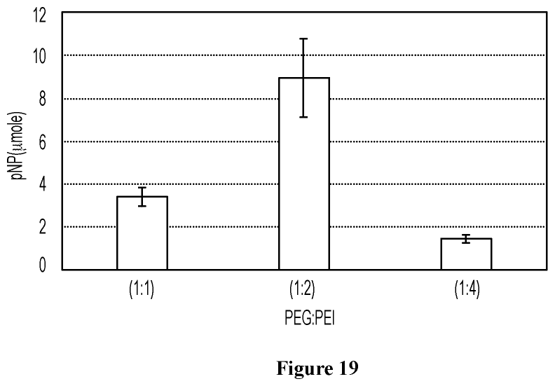

The matrices were functionalized by the polyethylene glycol (PEG) and polyethyleneimine (PEI) PEG-PEI copolymerization process at room temperature, followed by biotinylation. In contrast to previous PEG-PEI copolymerization processes (Star et al. (2003), Nano Letters 3 (4), 459-463, DOI: 10.1021/n10340172), ratios of PEG to PEI of greater than 1:1 but less than 1:4 were used in certain experiments and were shown to support increased enzyme activity (FIG. 19). A PEG:PEI ratio of 1:2 provided an IEC with more enzymatic activity than the 1:1 ratio or a 1:4 ratio in these experiments. To treat 50 mg of matrix material, 0.1 g of PEG and 0.2 g of PEI was used. The matrices including multiwall carbon fibers, agarose, carbon, polystyrene, silica, or nylon were first submerged in a 10 wt % solution of PEI (average molecular weight ˜25,000, Sigma-Aldrich, St. Louis, Mo.) and PEG (average molecular weight 10,000, Sigma-Aldrich, St. Louis, Mo.) in water overnight at room temperature followed by thorough rinsing with water and baking. Following the functionalization process, the amine-functionalized matrices (5 mg/ml) were conjugated via its exposing amine groups to biotin using the biotin 3-sulfo-N-hydroxysuccinimide ester (Sigma-Aldrich, St. Louis, Mo.).

### Example 2

### Enzymatic Conversion for Biofuel Production

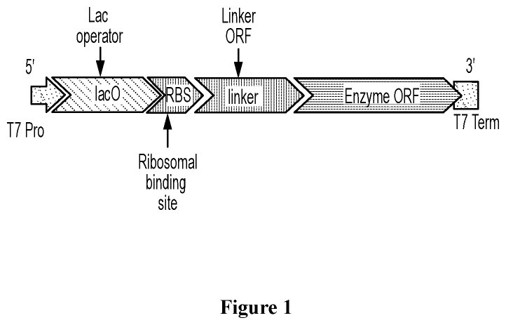

Construction of the gene expression vector for expression of streptavidin-fused enzymes—The gene expression vector for streptavidin-fused enzyme expression has been successfully constructed (FIG. 1). The gene of ionic liquid resistant and thermophilic cellulases, e.g., CelA3, β-1,4-endoglucanase (Cel5A) from Thermoanaerobacter tengcongensis MB4 (Liang, C., Xue, Y., Fioroni, M. et al. Appl Microbiol Biotechnol (2011) 89: 315. doi:10.1007/s00253-010-2842-6), endo-cellulase from Aspergillus niger, endoglucanase and 1,4-beta-cellobiosidase from Paenibacillus spp., endoglucanase II of Trichoderma reesei QM9414 (ATCC26921) (except the signal peptide region) will be amplified by splicing overlapping extension PCR. The PCR fragments of the gene will be cloned in a designed streptavidin-encoding open reading frame (ORF) built-in protein expression vector (pETstra) that is regulated by T7 expression system. The cellulase-cloned pETstra will be introduced into a BL21(DE3) E. coli strain that is specifically designed for expression of genes regulated by the T7 promoter.

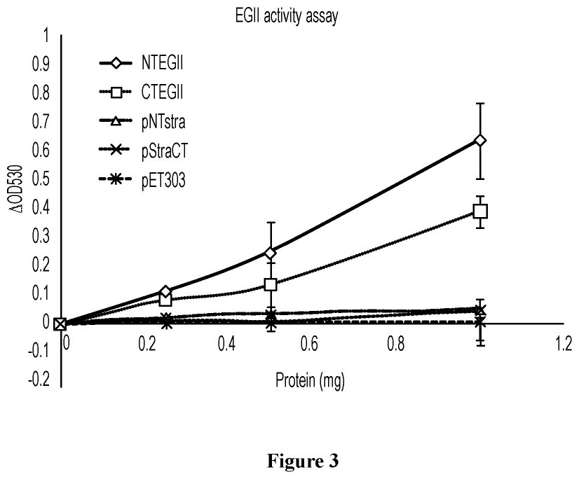

Expression and harvest of streptavidin-fused enzymes—An overnight culture of the BL21(DE3) E. coli carrying the EGII-cloned pETstra were prepared by inoculating a 10-ml LB with appropriate antibiotic with a single colony and incubating at 37° C. shaker. Two liters of LB with appropriate antibiotic were inoculated by adding 10 mL of overnight culture to each liter and incubating at 37° C. until the optical density at 600 nm reached 0.6-1.0. Then, IPTG was applied to the culture for the final concentration of 1 mM and the IPTG-induced culture was incubated at 22° C. for 18 hours. The protein-expressed culture was harvested by centrifugation at 5000 rpm for 10 min and the pellets collected and stored at −80° C. overnight. The pellets were re-suspended with PBS buffer and sonicated to break the cells. The sonicated prep was then centrifuged and the supernatant collected as crude extract. The protein concentration was measured by the Bradford method and enzymatic activity determined by a carboxymethylcellulose CMC-Congo red colorimetric assay using the measurement of the absorbance at 530 nm for Congo red for the hydrolysis of CMC by cellulases. Our preliminary results (FIG. 3) have demonstrated improved enzymatic activity when the streptavidin was fused at N-terminal as compared to C-terminal of the particular cellulases used in this example.

Immobilization of Streptavidin-fused Enzymes and Regeneration of the Polymer Platforms—The polymer matrices used in these experiments were multiwall carbon fibers that were derivatized by a PEI:PEG process essentially as described in Example 1. The streptavidin-fused enzymes, including endoglucanases, exoglucanases, and β-glucosidase, were then immobilized to the biotinylated matrices in the ratio of 1:5 to allow the strong streptavidin-biotin binding occurred (noncovalent interaction). Due to the strength and specificity of the interaction between streptavidin and biotinylated surface of matrices, it will allow immobilization of multi-enzyme complex in the continuous-flow bio-filter system but the expensive and labor-intensive enzyme purification will not be required (FIG. 2). The functionalized polymer matrices were rapidly regenerated by a simple thermal regeneration process. To date, six-cycles of regeneration have been performed. The matrix was regenerated by passing 80° C. of hot water for 10 min. Following the matrix regeneration process, fresh batch of streptavidin-fused enzymes was immobilized onto the functionalized polymer matrices again. This design allows the bio-filter cartridge to be replaced, regenerated with fresh enzyme, and reinstalled easily.

Chemical Analysis—To evaluate the conversion efficiency, enzyme stability/shelf-life, matrix regeneration cycles, cellulose depolymerization, sugar profiles of the immobilized engineered streptavidin-fused cellulases, including endoglucanases, exoglucanases, and β-glucosidase are determined.

The immobilized cellulases with supporting matrices (multiwall carbon fibers) were added to 5% (w/v) cellulose solutions with 5% of the SIGMACELL™ Microcrystalline Type 20 cellulose prepared in a 50 mM sodium acetate buffer (pH=5.0). Immediately after mixing, solutions were swirled and incubated at 3TC for exactly 120 min (2 hours). After incubation, the solutions were transferred into an ice bath to stop the reaction. The solutions were centrifuged at 3000 rpm for 10 minutes at 4° C. and the supernatants were collected for the sugars profiling and analysis.

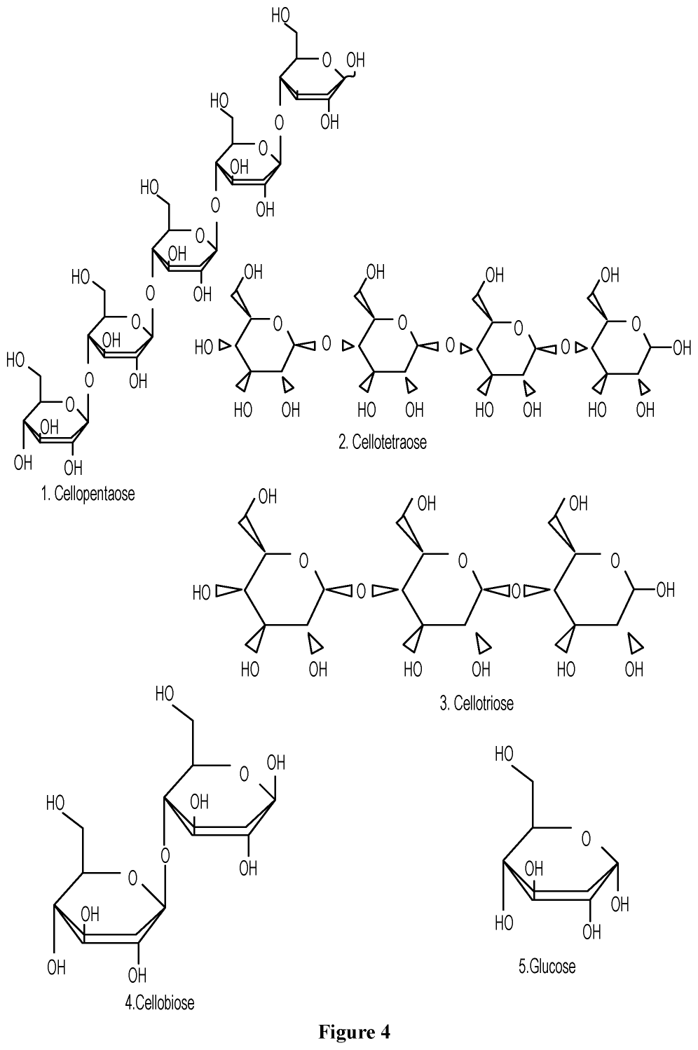

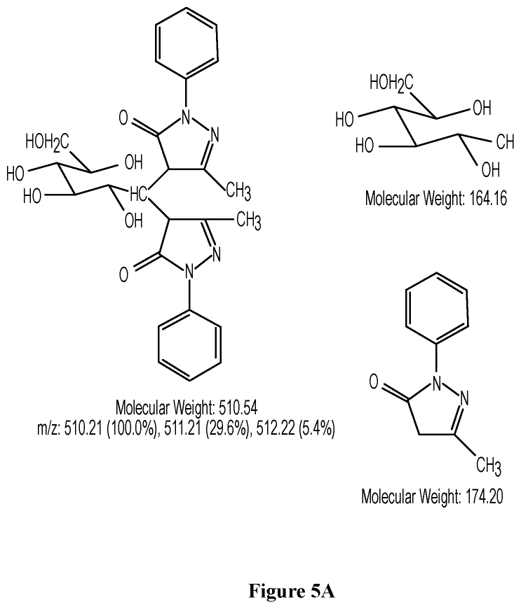

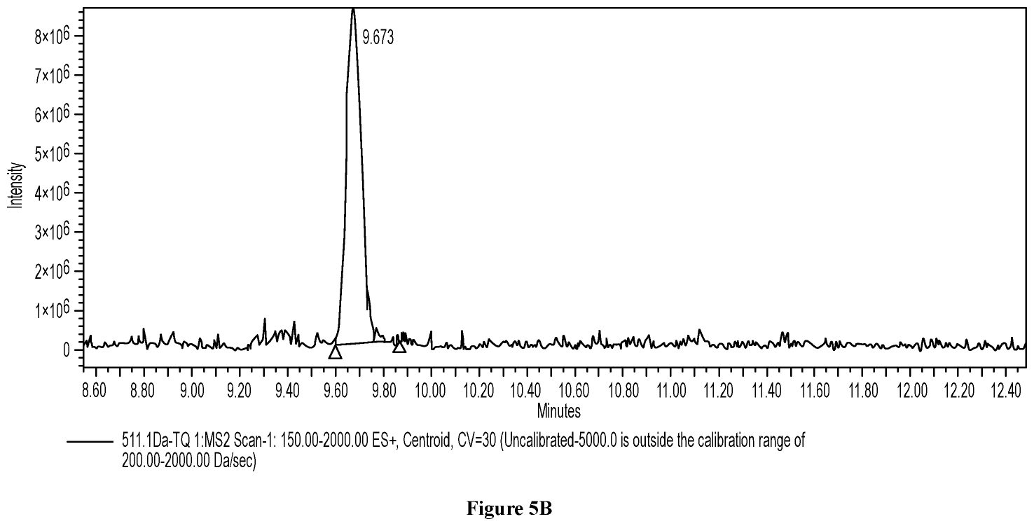

The formation of the sugar products and intermediates including glucose, cellobiose, cellotriose, cellotetraose, and cellopentaose were monitored by a Waters Alliance 2695 High Performance Liquid Chromatography system coupled with Waters ACQUITY™ TQD triple quadrupole mass spectrometer (HPLC-MS/MS). In this analytical process, 150 μL of the supernatants were first derivatized with 100 μL of 0.5 M 3-methyl-1-phenyl-5-pyrazolone (PMP) prepared in 0.5 N of NaOH. The derivatization solutions were heated at 70° C. for 30 min until the reaction was completed. The derivatized solution was neutralized with 0.3 N HCl and diluted with 1.65 mL of MeOH. Following the derivatization process, the PMP-derivatized sugars were separated and analyzed by a Waters Alliance 2695 reverse-phase HPLC equipped with a silica-based PHENOMENEX™ Columbus C8 column (4.6 mm by 150 mm, 5 μm; PHENOMENEX™, Torrance, Calif.). The mobile phase includes: (A) 100 mM ammonium acetate with 0.1% formic acid and (B) ACN with flow rate: 0.8 ml/min. The MS/MS system was operated using electrospray ionization (EI) in the positive ion mode with capillary voltage of 1.5 kV (ES-). The ionization source was programmed at 150° C. and the desolvation temperature was programmed at 450° C. The molecular parent ions were screened and the product ions used for the quantifications were determined from the spectra obtained from injecting 30 μL of a standard solution containing 1000 μg/L of the analytical standards. Analytical data were processed using Waters Empower software (Waters, Calif., USA). The detailed retention times and selected quantification ions for each sugar were described as in Table 1 and FIGS. 4 and 5.

TABLE 1
The retention times and selected quantification ions for analysis
of PMP-sugars by HPLC-MS.

| Sugars | Retention Time | Quantification Ions (m/z) |
|---|---|---|
| Glucose | 9.69 | 511 |
| Cellobiose | 9.43 | 673 |
| Cellotriose | 9.27 | 835 |
| Cellotetraose | 9.17 | 997 |
| Cellopentaose | 9.11 | 1160 |

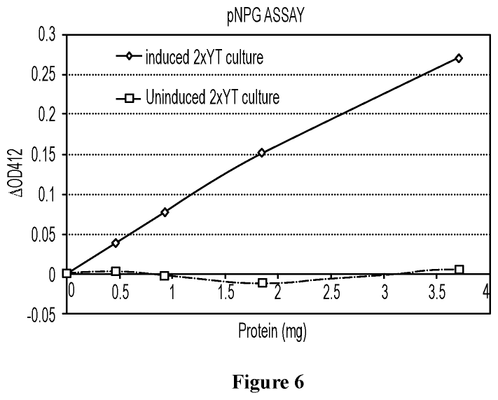

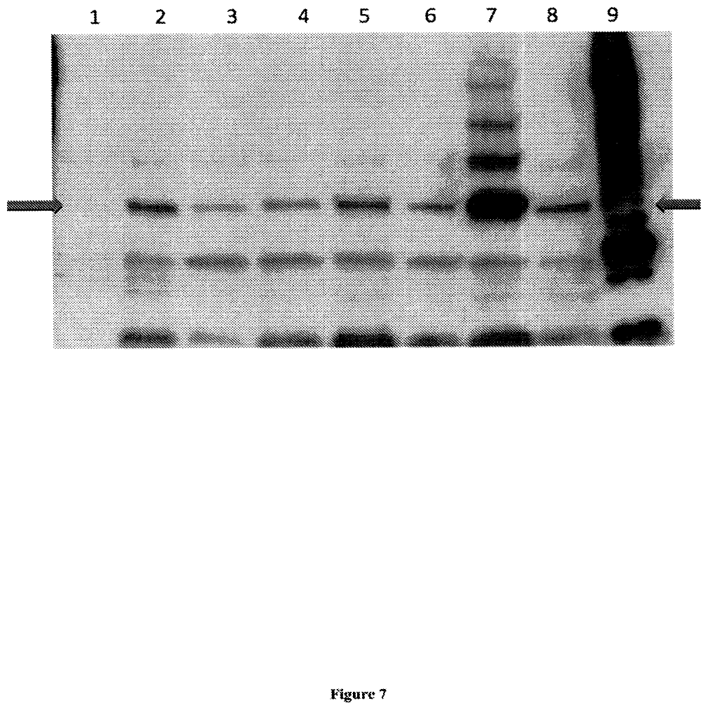

The expression of streptavidin-fused cellulases was performed in the E. coli cultures containing the expression vectors and the crude extract of the cultures were processed and tested for the enzymatic activity of the protein. The expression vectors contain egII ORF with streptavidin fused either at the N-terminus or at the C-terminus expressed the enzymatic activity of EGII. The expression vectors without egII ORF inserted showed none of enzymatic activity. (FIG. 3) The expression of streptavidin-fused cellulases was controlled by IPTG induction. The crude extract from the culture without IPTG induction showed no enzymatic activity of β-glucosidase, using p-nitrophenyl β-D-glucopyranoside as substrate, in comparison to the sample from the IPTG induced culture. (FIG. 6). The crude extract from several culture preparations was adjusted to equal amounts of proteins, electrophoresed and transferred to a blot, using anti-streptavidin monoclonal antibody to detect the presence of streptavidin-fused protein in the samples. (FIG. 7)

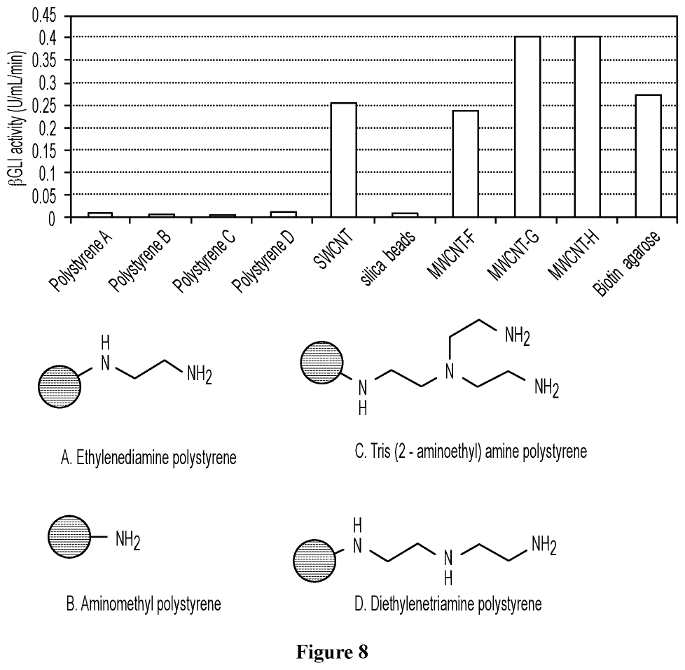

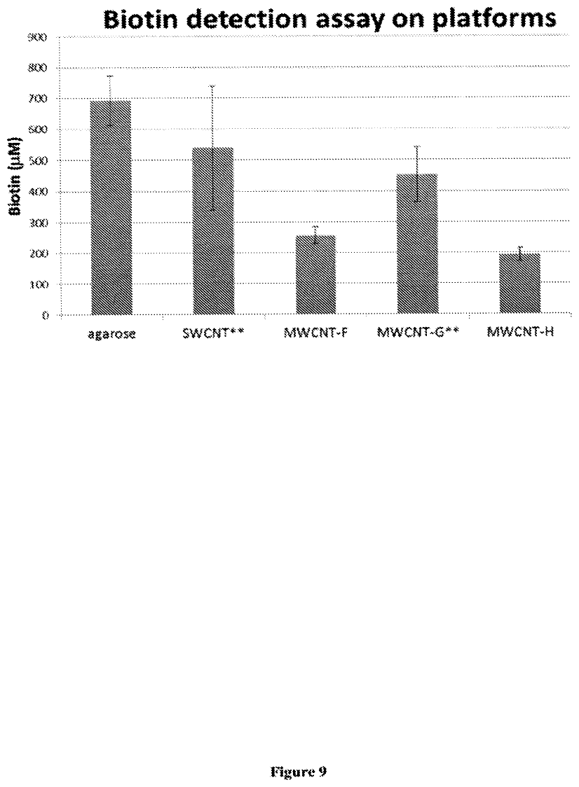

The low cost and easy PEG-PEI copolymerization process rapidly provides the primary amine group (NH₂) required for the following biotinylation reaction. Among the selected polymer supporting material used in these experiments, the biotinylated multiwall carbon nanomaterial (MWCNT-G, 5-9 μm), multiwall carbon nanomaterial (MWCNT-H, 2.5-20 μm) have the best capacity to immobilize the streptavidin-fused cellulase (β-glucosidase), followed by biotinylated agarose, single wall carbon nanomaterial (SWCNT 0.7-1.3 nm) and multiwall carbon nanomaterial (MWCNT-F, 0.5-10 μm) (FIG. 8). The concentrations of biotin were confirmed and quantified (FIG. 9).

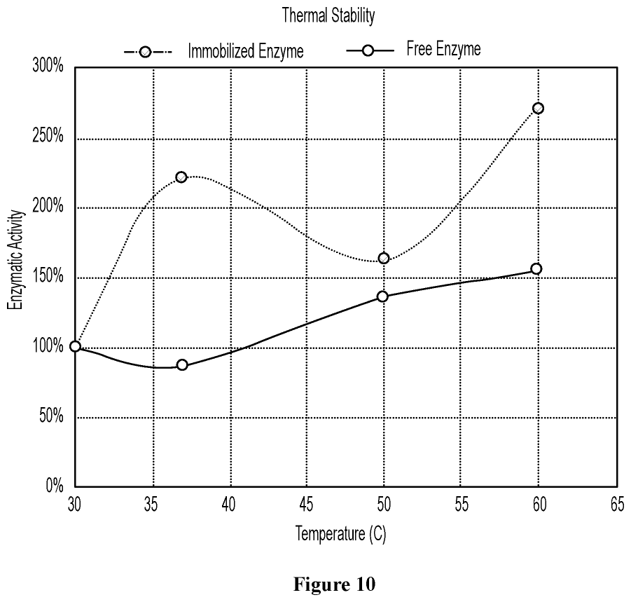

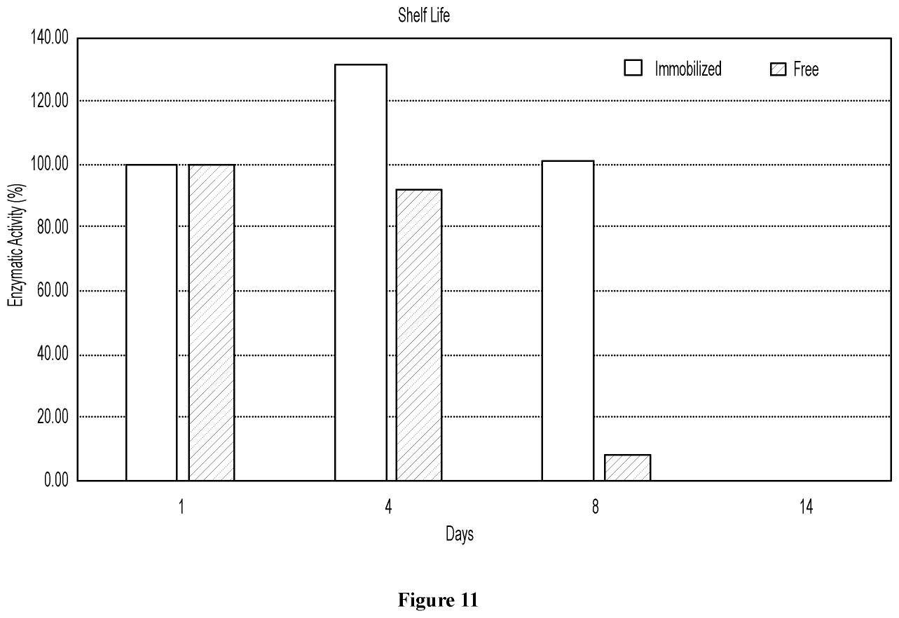

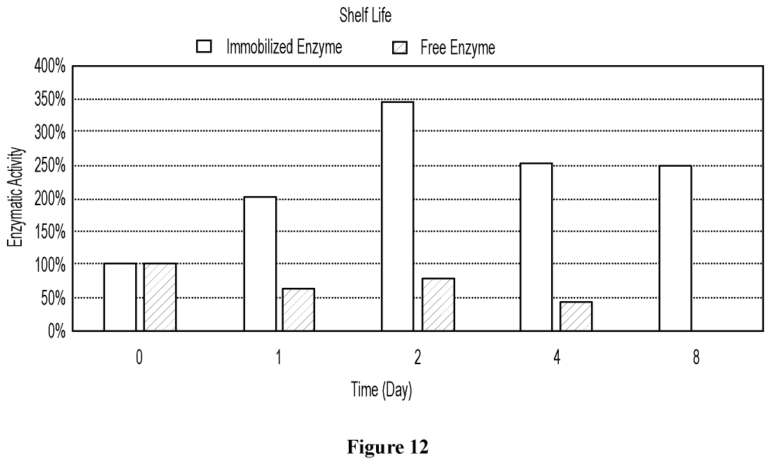

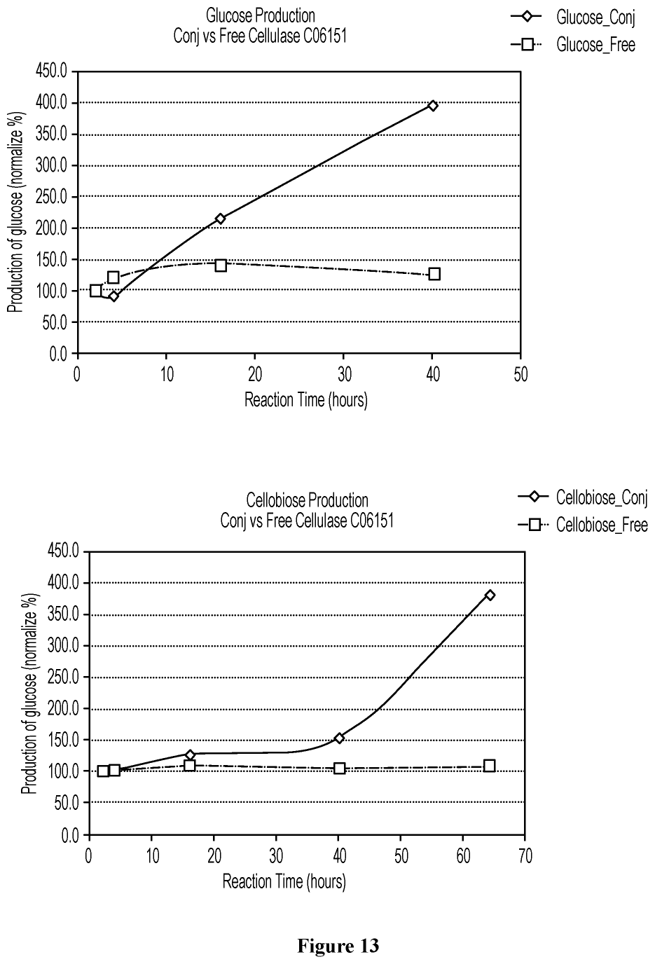

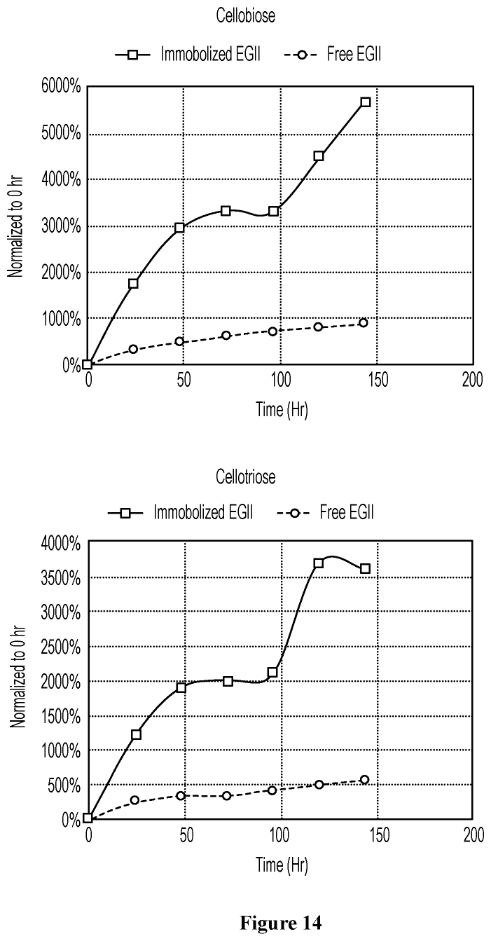

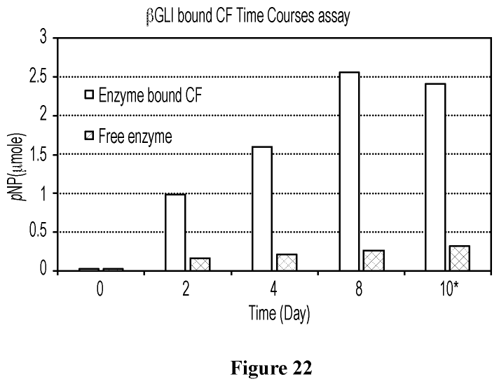

The results of the time course experiments have shown increased enzymatic stabilities when the cellulases were immobilized onto biotinylated silica, agarose, and carbon matrices (FIGS. 11 and 12). The immobilized cellulases have shown increased thermal stability as compared to the free enzyme (FIG. 10) and the shelf life of the cellulases was increased from 4 days to 8 days when they were immobilized to the supporting matrices (FIGS. 11 and 12). As a result of the enhanced stability of the immobilized enzymes, the production of sugars as compared to free enzyme was increased by around 400%-700% over 70-120 hours reaction time (FIGS. 13, 14 and 22) and was increased even higher after longer periods of testing (FIG. 22).

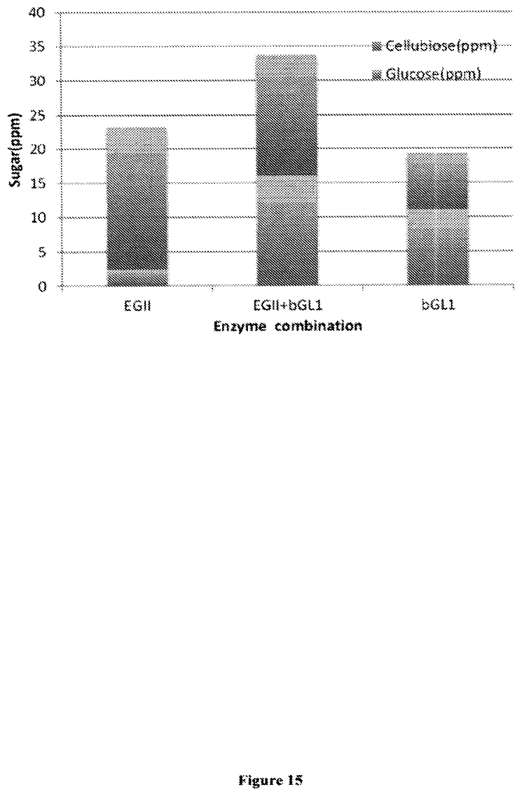

The conversion efficiencies were further improved when multi-enzyme complexes were developed by the method herein. The immobilization of both streptavidin-fused endoglucanase and β-glucosidase on the same platform enhances the production of glucose by 133-530% as compared to either of the immobilized enzyme alone (FIG. 15).

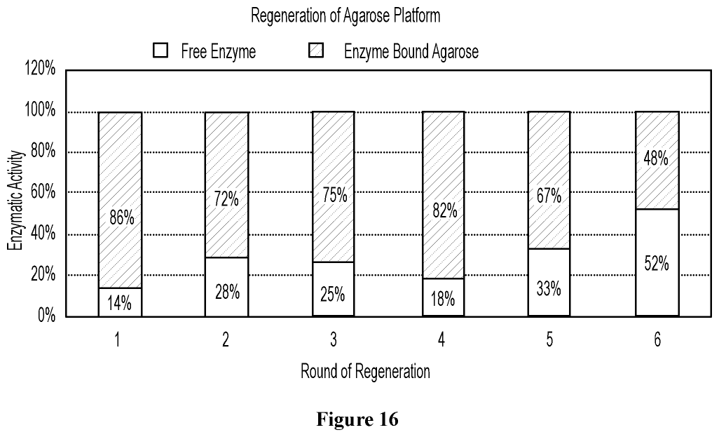

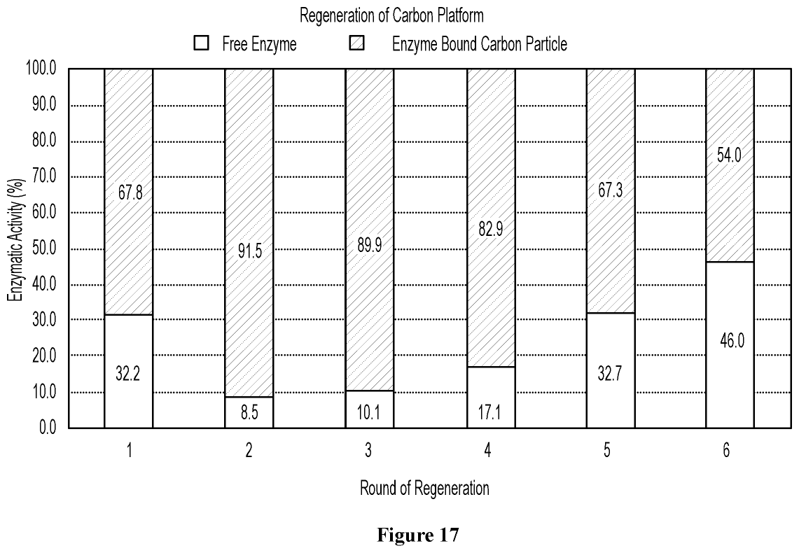

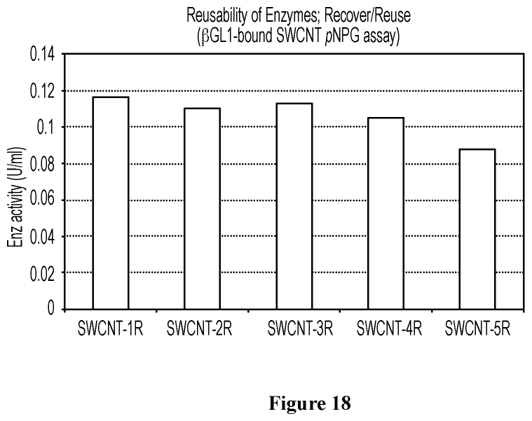

The matrices have been successfully regenerated up to 6 times (still ongoing) with a simple thermal regeneration process by treating the matrix with 80° C. water for 10 min (FIGS. 16 and 17). The results suggested that the regeneration cycles did not significantly degrade the functionalized surface on the matrices. For the agarose matrix, about 86.17% of the recombinant enzyme could be attached to the agarose after the first round of regeneration. The enzymatic activity was maintained above 72% after 2-4 regeneration cycles, and then it decreased below 50% in the 6th regeneration (FIG. 17). The regeneration experiment has been stopped due to agarose degradation after the 6th heat de-attachment. For the carbon matrix, the highest enzyme bound to carbon particles was 91.5% in the 2nd round. The activity of enzyme bound was maintained above 50% up to six regeneration cycles, similar to the results with agarose matrix. The carbon matrix exhibits high thermal stability (FIG. 18).

### Example 3

### Blood Type Conversion

In another example, this technology has been used for the conversion of blood types. Blood cannot be manufactured; it can only come from generous donors. Most donated red blood cells must be used within 42 days of collection or discarded. The blood type most often requested is Type O Rh negative blood (red cells) that can be transfused to patients of all blood types. Type O is always in great demand and often in short supply. Type O Rh negative blood, the universal blood, is needed in emergencies for who need blood immediately before their blood type is known. As noted, there have been prior efforts to produce type O blood utilizing enzymatic processes to cleave off the A or B immunodominant sugar of blood group A or B red blood cells. However, current enzymatic conversion technologies often require extensive centrifugation and wash steps, prior to achieve optimal condition for enzymatic conversion and post to remove the enzyme residues from blood before transfusion to meet blood storage condition for transfusion standard protocols. These prior and post conversion processes resulted in a serious concern that converted red blood cells (ECO RBC) would be damaged and fragile. The survival rate of ECO RBC dropped to 70% or less after the process. In contrast, the IEC does not require extensive wash and centrifugation steps to assure the completion of antigen removal that avoids losing red blood cells in the process. The system eliminates enzyme residues from converted Type O universal blood, therefore completely eliminating the risk of the immunoreaction.

The continuous flow system can comprise of recombinant enzymes genetically fused with a protein that specifically binds to the functionalized surface of a readily regenerated bio-filter system. The immobilized recombinant exoglycosidases in the system, such as alpha-N-acetylgalactosaminidase or alpha-galactosidase, remove N-acetylgalactosamine or galactose residues, respectively, at the non-reducing end of carbohydrate precursor chain and convert A or B antigen into H antigen, thus producing group O red blood cells. With the continuous flow system, the production of enzymatically converted universal red blood cells can be guaranteed without applying excess enzyme.

A Clostridium perfringens alpha-N-acetylgalactosaminidase enzyme that converts Type A Rh negative blood to universal Type O blood was first identified in 2000 (Hsieh, H.-Y., et al.. (2000), IUBMB Life, 50: 91-97. DOI: 10.1080/713803702). An aagA gene from Clostridium perfringens encoding alpha-N-acetylgalactosaminidase was PCR amplified and fused with the designed streptavidin gene. The fusion gene fragments were cloned into pET303, a commercial T7 expression vector purchased from Invitrogen. The clone was expressed in BL21(DE3) RIL E. coli host and induced by IPTG for protein production. The recombinant AagA streptavidin fusion protein was isolated essentially as described in Example 2 and applied to a biotin functionalized carbon fiber matrix prepared essentially as described in Example 1 for immobilization. The carbon fibers used in the PEG:PEI derivatization methods were ¼″ graphite fibers (Part # 571; Fibre Glast Development Corporation, Brookville, Ohio).

About 5% of type A red blood cell suspension was prepared in CPD solution and placed in the sterile bag containing AagA-immobilized carbon fibers. The conversion reaction was incubated at 25° C. with agitation for 2 hours. The converted cell suspension was collected by pouring out of the bag. A 1-mL subsample from the converted cell suspension was immunolabeled with monoclonal anti-A antibody or anti-H antibody, then conjugated with Alexa 488 anti-mouse IgG and sent for flow cytometry assay. The results showed the decrease of anti-A antibody and the increase of anti-H antibody detected in the converted cells that proved type A blood cells were converted to type O by our enzyme-immobilized matrix.

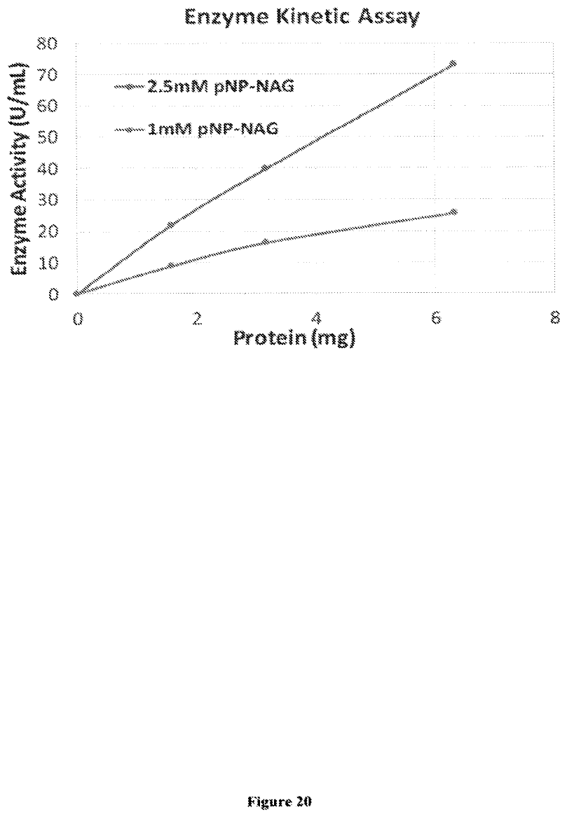

Our system has successfully demonstrated the specific activity of immobilized □-N-acetylgalactosaminidase determined by quantifying the hydrolysis of p-nitrophenol from 4-Nitrophenyl N-acetyl-a-D-galactosaminide (N4264, Sigma-Aldrich, St. Louis, Mo.) (FIG. 20).

### Example 4

### Industrial Blood Conversion

Group A RBCs will undergo enzymatic conversion using a recombinant Clostridium perfringens α-N-acetylgalactosaminidase, any of SEQ ID NO: 5 through SEQ ID NO: 10, an alpha-galactosidase, or a combination thereof that are immobilized on the functionalized matrix. The process of enzymatic conversion will be carried out by aseptic techniques in a sealed container. The RBC component will be centrifuged to remove supernatant plasma and the packed RBCs will be then resuspended in an isotonic phosphate-citrate-sodium chloride buffer (pH 6.5-7.0) or one of FDA-approved blood preservative solutions. The RBC preparation will be added into the sterile container containing the IEC. The enzymatic conversion will be incubated either at room temperature or at cold room. ECO RBCs will be drained out of the converter and collected in a sterile container. Converted RBC units will be stored at 1° C. to 6° C.

Removal of A or B antigen will be confirmed by immunolabeling with anti-A or anti-B murine mAb and Alexa 488 secondary conjugates followed by flow cytometry to determine the efficiency of enzymatic conversion. The immunolabeling procedure will be performed in the biosafety cabinet.

In certain cases, complete conversion is expected from our IEC incorporated continuous flow system; red blood cell suspensions will be circulated through the enzymatic blood converter to enhance the efficiency of the conversion in a short period of time.

### Example 5

### Production of Biodiesel

In another example, an IEC will be used for the production of Biodiesels. The annual world consumption of diesel is approximately 934 million tons, of which Canada and the United States consume 2.14 and 19.06%, respectively (Marchetti, et al. (2008), Fuel Process. Technol., 89: 740-748. DOI: 10.1016/j.fuproc-2008-01-007). Most of the oils currently are made from soybeans, palm or rapeseed. The enzymatic process is known to be a clean and environment friendly technique for biodiesel production. This process can simultaneously convert both free fatty acids and triglyceride into biodiesel. This IEC will allow production of multi-enzyme system immobilized with a wide range of lipases, such as Rhizopus oryzae lipases, Candida rugosa lipases, and lipases of SEQ ID NO: 19 through SEQ ID NO: 26 or variants thereof to facilitate the enzymatic transesterification process for production of biodiesel.

### Example 6

### Production of Specialty Chemicals

In another example, the IEC will be used for the production of specialty chemicals. Since 2000, more than 100 different enzymatic biocatalytic processes have been implemented in pharmaceutical, chemical, agricultural, and food industries. The advantages of this green biocatalytic process over the traditional chemical processes include lower cost, higher product purity, and elimination of the toxic chemicals in the manufacture process and waste. The enzymatic process also significantly reduces the number of synthetic steps that would be required for conventional synthesis. Several classes of enzymes including ketoreductases, transaminases, amine oxidases, mono-oxygenases and acyl transferases, have been utilized for a wide range of common chemical conversions in the manufacture process of pharmaceuticals and specialty chemicals such as Telaprevir (Telavic, INCIVEK™), Sitagliptin (JANUVIA™) Simvastatin (Lipovas, ZOCOR™), Atazanavir (REYATAZ™), Esomeprazole (NEXIUM™), Atorvastatin (LIPITOR™), Montelukast (SINGULAIR™), Boceprevir (VICTRELIS™), and S-methoxyisopropylamine. In the food industry, enzymes, such as amyloglucosidase and amylase glucose isomerases, have been used to produce fructose syrups (sweeteners) from corn starch. This IEC will be utilized to produce multi-enzyme systems with immobilized ketoreductases, transaminases, amine oxidases, mono-oxygenases or acyl transferases to increase the yield and purity and eliminate the toxic chemicals in the production process.

### Example 7

### Wound Healing Patch or Spray (e.g., Proteolytic Enzymes)

In another example, the IEC will be used to develop wound healing patches or sprays utilizing proteolytic enzymes, produced and immobilized on matrices that were functionalized essentially as indicated in Example 2. Wound healing is a multi-factorial physiological process. Several enzymatic pathways become active during repair and help the tissue to heal. The IEC will be used to express and immobilize the antimicrobial enzymes, peptide, or complex, such as GLG-enzyme complex (glucose oxidase combined with lactoperoxidase) on a wound healing patch. The PEG used in the process outlined herein is a biocompatible polymer with low immunogenicity. Immobilized enzymes used in the IEC in the wound healing patch could also include proteases such as papain or a collagenase.

Previous studies have shown that proteolytic enzymes such as papain immobilized in pectin, can be used for the development of effective aerosol spray system for wound healing in the areas of enzymatic debridement of necrotic tissue and liquefaction of slough. This process will help to remove dead or contaminated tissue in acute and chronic lesions, such as diabetic ulcers, pressure ulcers, varicose ulcers, and traumatic infected wounds, postoperative wounds, burns, carbuncles, and pilonidal cyst wounds (Júregui et al. (2009), Biotechnology and Bioprocess Engineering. 14: 450-456, DOI: 10.1007/s12257-008-0268-0.). The IEC provided herein can be used to stabilize the proteolytic enzymes in the aerosol spray.

### Example 8

### Drug Delivery Systems (e.g., Antimicrobial Proteins: Lysozyme etc.)

In another example this IEC system will be used to deliver drugs such as antimicrobial proteins for various practical applications. The IEC will be used as platforms to deliver antimicrobial proteins, peptides, or antibodies for therapeutic purposes. For example, lysozyme has been demonstrated to have antibacterial activity against organisms, including Listeria monocytogenes and certain strains of Clostridium botulinum. The immobilized antimicrobial enzymes like lysozyme, lactoferrin or their complex will be used for e.g. disinfection products or food packaging (food safety).

### Example 9

### Development of Magnetic Enzymatic Biocatalyst System

In another example, this enzymatic platform technology will be used to develop a low-cost and recoverable magnetic nanobiocatalyst system. The advantages of the system include high surface area, biocompatibility, a modifiable surface and easy recovery. The magnetic nanobiocatalyst can be easily recovered by applying an external magnetic field. Enzymes will be fused to streptavidin as indicated in prior Examples.

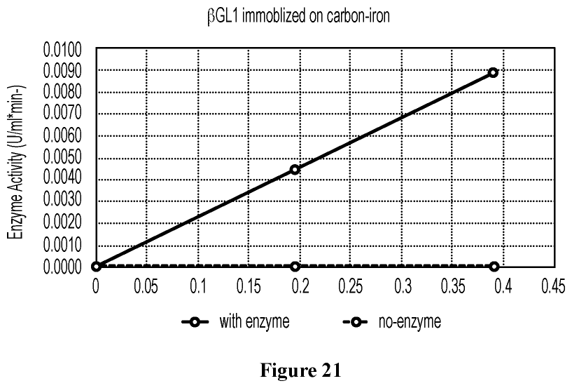

Cellulases (β-glucosidase) fused with streptavidin have been successfully immobilized onto the functionalized magnetic carbon-ion nanoparticles (FIG. 21). The functionalization process involved 1) sonication of 2 mg MWCNTs in 10 mL of toluene solution with 0.1% (v/v) oleylamine for 2 hours, 2) washing oleylamine functionalized MWCNTs with ethanol, 3) dispersion in toluene, and 4) addition of the magnetic iron oxide nanoparticles and hexane into the reaction followed by mildly sonication for 5 mins. The magnetic carbon-ion nanobiocatalysts have been successfully recovered by applying an external magnetic field (FIG. 21).

### Example 10

### Enzyme Protein Sequences and DNA Sequence

TABLE 2
Protein Sequences and SEQ ID NO: 35 DNA sequence

| SEQ ID | Streptavidin | Streptavidin mature peptide |
|---|---|---|
| NO: 1 | mature peptide | |
| SEQ ID | ATCC26921 | βGLI of Trichoderma reesei QM9414 without signal peptide: |
| NO: 2 | | |
| SEQ ID | ATCC26921 | EGII of Trichoderma reesei QM9414 without signal peptide: |
| NO: 3 | | N |
| SEQ ID | ATCC10543 | AagA of Clostridium perfringens: |
| NO: 4 | | |
| SEQ ID | UniProtKB/ | Alpha-D-galactoside galactohydrolase of Coffea arabica (coffee) (mature |
| NO: 5 | Swiss-Prot: | sequence): |
| | Q42656.1 | |
| SEQ ID | GenBank ID: | NAGA gene for alpha-N-acetylgalactosaminidase from Elizabethkingia |
| NO: 6 | AM039444.1 | meningoseptica comb. nov. |
| SEQ ID | GenBank ID: | NAGA gene for alpha-N-acetylgalactosaminidase from Bacteroides fragilis, |
| NO: 7 | AM039447.1 | strain ATCC25285D, clone 2 |
| SEQ ID | GenBank ID: | NAGA gene for alpha-N-acetylgalactosaminidase from Shewanella |
| NO: 8 | AM039445.1 | oneidensis strain ATCC70050 |
| SEQ ID | GenBank ID: | NAGA gene for alpha-N-acetylgalactosaminidase from Tannerella forsythia |
| NO: 9 | AM039448.1 | comb. nov., strain ATCC43037 |
| SEQ ID | GenBank ID: | alpha-galactosidase A (partial sequence) from Bacteroides fragilis, strain |
| NO: 10 | EXY33367.1 | 3397 T10 |
| SEQ ID | JF826525 | CelA3 endoglucanase (metagenomic) |
| NO: 11 | | |
| SEQ ID | JF802029 | a Cel5K endoglucanase (metagenomic) |
| NO: 12 | | |
| SEQ ID | Gene ID: | Endoglucanase of Paenibacillus odorifer (strain DSM_15391) |
| NO: 13 | 31573201; | |
| | NCBI: | |
| | WP_03857297 | |
| | 2.1 | |
| SEQ ID | Gene ID: | Endoglucanase (hypothetical protein) of Paenibacillus odorifer |
| NO: 14 | 31571570; | |
| | NCBI: | |
| | WP_05209705 | |
| | 4.1 | |
| SEQ ID | Gene ID: | Paenibacillus 1,4-beta-cellobiosidase |
| NO: 15 | 31573200 | |
| | NCBI: | |
| | WP_08074272 | |
| | 5.1 | |
| SEQ ID | Gene ID: | endo-1,4-beta-xylanase A from Thermotoga maritima MSB8 |
| NO: 16 | 896885; NCBI: | |
| | NP_227877.1 | |
| SEQ ID | Gene ID: | alpha-L-arabinofuranosidase A-like protein from Bifidobacterium |
| NO: 17 | 31840121; | thermophilum RBL67 |
| | NCBI: | |
| | WP_01545074 | |
| | 3.1 | |
| SEQ ID | NP_229549 | Endoglucanase Tma_Cel5A from Thermotoga maritama |
| NO: 18 | | |
| SEQ ID | UniProtKB: | Rhizopus oryzae lipase (ROL) |
| NO: 19 | B1Q560_RHIO | |
| | R | |
| SEQ ID | Gene ID: | Lipase of Oryza sativa Japonica |
| NO: 20 | 4343234; | |
| | NCBI: | |
| | XP_015644618 | |
| | .1 | |
| SEQ ID | GenBank: | Lipase of Diutina rugosa (Candida rugosa) |
| NO: 21 | ACN78942.1 | |
| SEQ ID | UniProtKB: | LIP1, Lipase 1 of Diutina rugosa (Candida rugosa) |
| NO: 22 | P20261 | |
| SEQ ID | UniProtKB: | LIP2, Lipase 2 of Diutina rugosa (Candida rugosa) |
| NO: 23 | P32946 | |
| SEQ ID | UniProtKB: | LIP 3, Lipase 3 of Diutina rugosa (Candida rugosa) |
| NO: 24 | P32947 | |
| SEQ ID | UniProtKB: | LIP4, Lipase 4 of Diutina rugosa (Candida rugosa) |
| NO: 25 | P32948 | |
| SEQ ID | UniProtKB: | LIPS, Lipase 5 of Diutina rugosa (Candida rugosa) |
| NO: 26 | P32949 | |
| SEQ ID | UniProtKB: | Atrazine chlorohydrolase (AtzA) from Pseudomonas sp. (strain ADP) |
| NO: 27 | P72156 | |
| SEQ ID | UniProtKB: | Hydroxydechloroatrazine ethylaminohydrolase (AtzB) from Pseudomonas |
| NO: 28 | P95442 | sp. (strain ADP) |
| SEQ ID | UniProtKB: | N-isopropylammelide isopropyl amidohydrolase (AtzC) from Pseudomonas |
| NO: 29 | 052063 | sp. (strain ADP) |
| SEQ ID | UniProtKB: | Cyanuric acid amidohydrolase (AtzD) from Pseudomonas sp. (strain ADP) |
| NO: 30 | P58329 | |
| SEQ ID | UniProtKB: | Biuret hydrolase (AtzE) from Pseudomonas sp. (strain ADP) |
| NO: 31 | Q936X3 | |
| SEQ ID | UniProtKB: | Allophanate hydrolase (AtzF) from Pseudomonas sp. (strain ADP) |
| NO: 32 | Q936X2 | |
| SEQ ID | GenBank | PnrA Nitroreductase from Pseudomonas putida |
| NO: 33 | Protein ID: | |
| | SKB88864.1 | |
| SEQ ID | NCBI: | β-1,4-endoglucanase (Cel5A) from Thermoanaerobacter tengcongensis MB4 |
| NO: 34 | WP_01102480 | (Liang, C., Xue, Y., Fioroni, M. etal. Appl Microbiol Biotechnol (2011) 89: |
| | 8.1 | 315. doi:10.1007/s00253-010-2842-6) |
| SEQ ID | NCBI: | DNA encoding the SEQ ID NO: 4 β-1,4-endoglucanase (Cel5A) from |
| NO: 35 | WP_01102480 | Thermoanaerobacter tengcongensis MB4 (Liang, C., Xue, Y., Fioroni, M. et |
| | 8.1 | al. Appl Microbiol Biotechnol (2011) 89: 315. doi:10.1007/s00253-010- |
| | | 2842-6) |

The breadth and scope of the present disclosure should not be limited by any of the above-described exemplary embodiments, but should be defined only in accordance with the following claims and their equivalents.

### REFERENCES

1. Shen-Long, T. Park, and W. Chen, Size-modulated synergy of cellulase clustering for enhanced cellulose hydrolysis. Biotechnol. J., 2013. 8: p. 257-261.

2. Oswald, P. R., et al., Properties of a thermostable β-glucosidase immobilized using tris(hydroxymethyl)phosphine as a highly effective coupling agent. Enzyme and Microbial Technology, 1998. 23: p. 14-19.

3. Cochrane, F. C., H. H. Petach, and W. Henderson, Application of tris(hydroxymethyl)phosphine as a coupling agent for alcohol dehydrogenase immobilization. Enzyme and Microbial Technology, 1996. 18: p. 373-378.

4. Kim, D. M., Umetsu, M., Takai, K., Matsuyama, T, Enhancement of cellulolytic enzyme activity by clustering cellulose binding domains on nanoscaffolds. 2011. 7: p. 656-664.

5. Afsahi, B., et al., Immobilization of Cellulase on Non-Porous Ultrafine Silica Particles. Scientia Iranica, 2007. 14(4): p. 379-383.

6. Wyman, C. E., Handbook on Bioethanol Production and Utilization 1996: Taylor & Francis

7. Yuan, X., et al., Immobilization of cellulase using acrylamide grafted acryloni-tride copolymer membranes. Journal of Membrane Science, 1999. 155: p. 101-106.

8. Ohison, I., G. Tragardh, and B. Hahn-Hagerdal, Enzymatic hydrolysis of sodium hydroxide pretreated sallow in an ultrafltration membrane reacto. Biotechnology & Bioengineering, 1984. 26: p. 647-653

9. Henley, R. G., R. Y. K. Yang, and P. F. Greenfeld, Enzymatic saccharification of cellulose in membrane reactors. Enzyme Microbiology & Technology, 1980. 2: p. 206-208

10. Tjerneld, F., et al., Enzyme cellulose hydrolysis in an attrition bioreactor combined with an aqueous two-phase system. Biotechnology & Bioengineering, 1991. 37: p. 876-882.

11. Tjerneld, F., et al., Enzyme recycling in cellulose hydrolysis combined use of aqueous two-phase systems and ultrafiltration. Biotechnology & Bioengineering Symp., 1985. 15: p. 419-429

12. Park, J. W. and T. Kajiuchi, Development of effective modified cellulase for cellulose hydrolysis process. Biotechnology & Bioengineering, 1995. 45: p. 366-373.

13. Jochems, P., et al., Enzyme immobilization on/in polymeric membranes: Status, challenges and perspectives in biocatalytic membrane reactors (BMRs). Green Chem., 2011. 13: p. 1609-1623.

14. Lamed, R. and E. A. Bayer, The cellulosome of Clostridium thermocellum. Adv. Appl. Microbiol., 1988. 33: p. 1-46.

What is claimed is:

1. An immobilized enzyme complex (IEC) comprising a heat stable matrix that is covalently attached to a biotin molecule or analog thereof with a linker molecule and a fusion protein comprising an enzyme domain and a biotin binding domain (BBD), wherein the biotin binding domain is non-covalently bound to the biotin molecule or analog thereof.

2. The immobilized enzyme complex of claim 1, wherein said heat stable matrix comprises carbon fiber, polystyrene, polylactic acid, polyurethane, silica, nylon, or polypropylene.

3. The immobilized enzyme complex of claim 1, wherein said heat stable matrix is selected from the group consisting of carbon fiber, polystyrene, polylactic acid, polyurethane, silica, nylon, and polypropylene.

4. The immobilized enzyme complex of claim 1, wherein the heat stable matrix is at least partially coated with a mixture of polyethylene glycol (PEG) and polyethyleneimine (PEI).

5. The immobilized enzyme complex of claim 1, wherein the linker molecule is attached to polyethyleneimine (PEI) molecules coating the matrix.

6. The immobilized enzyme complex of claim 1, wherein said heat stable matrix does not comprise a magnetic particle.

7. The immobilized enzyme complex of claim 1, wherein said biotin analog comprises desthiobiotin, 2′-iminobiotin, biotin sulfone, bisnorbiotin, tetranorbiotin, oxybiotin, any derivative thereof, or any derivative of biotin that can be bound by the BBD.

8. The immobilized enzyme complex of claim 5, wherein said linker molecule comprises an alkane, an alkyl group, an amide, or combination thereof.

9. The immobilized enzyme complex of claim 1, wherein the enzyme domain is selected from the group consisting of a hydrolase, ketoreductase, transaminase, amine oxidase, mono-oxygenase, and an acyl transferase domain.

10. The immobilized enzyme complex of claim 1, wherein the enzyme domain is fused either to the N-terminus of the BBD or to the C-terminus of the BBD.

11. The immobilized enzyme complex of claim 10, wherein the enzyme domain is fused to the BBD with a peptide linker.

12. The immobilized enzyme complex of claim 1, wherein the enzyme domain is a glycoside hydrolase domain.

13. The immobilized enzyme complex of claim 12, wherein the glycoside hydrolase is an alpha-N-acetylgalactosaminidase, alpha-galactosidase, beta-glucosidase, a cellulase, an endoglucanase, or an exoglucanase.

14. The immobilized enzyme complex of claim 1, wherein at least two fusion proteins are immobilized on the matrix.

15. The immobilized enzyme complex of claim 14, wherein the fusion proteins comprise an enzyme domain that are each independently selected from the group consisting of a beta-glucosidase, an endoglucanase, and an exoglucanase.

16. The immobilized enzyme complex of claim 14, wherein at least one enzyme domain comprises a polypeptide having at least 70% sequence identity to a beta-glucosidase (SEQ ID NO: 2), an endoglucanase (SEQ ID NO: 3), an alpha N-acetylgalactosaminidase (SEQ ID NO: 4), an alpha-galactosidase (SEQ ID NO: 5), SEQ ID NO: 6-33, or SEQ ID NO: 34.

17. The immobilized enzyme complex of claim 1, wherein the BBD comprises an avidin BBD, streptavidin BBD, tamavidin BBD, zebavidin BBD, bradavidin BBD, rhizavidin BBD, shwanavidin BBD, xenavidin BBD, a chimera thereof, or derivative thereof having one or more amino acid residue insertions, deletions, or substitutions.

18. The immobilized enzyme complex of claim 1, wherein the IEC or matrix is biocompatible.

19. The immobilized enzyme complex of claim 1, wherein the enzyme domain has proteolytic activity.

20. The immobilized enzyme complex of claim 1, wherein the enzyme domain comprises a ketoreductase, transaminase, amine oxidase, mono-oxygenase, or acyl transferase domain.

21. The immobilized enzyme complex of claim 1, wherein the enzyme domain: (i) reduces RDX (hexahydro-1,3,5-trinitro-1,3,5-triazine); (ii) reduces 2,4,6-trinitrotoluene (TNT); (iii) reduces chromium 6+ to chromium 3+; or (iv) has 2,2′,3-trihydroxybiphenyl dioxygenase activity.

22. The immobilized enzyme complex of claim 21, wherein the IEC is contained in an enclosure that is permeable to a substrate and a product of the enzyme domain activity of (i), (ii), (iii), or (iv), and comprises the enzyme domain of (i), (ii), (iii), or (iv), respectively.

23. The immobilized enzyme complex of claim 22, wherein the matrix is carbon fiber.

24. The immobilized enzyme complex of any one of claims 1 to 23 that is contained in a bioreactor system or in an enclosure that is permeable to a substrate and a product of the enzyme domain-catalyzed conversion of the substrate.

25. The immobilized enzyme complex of any one of claims 1 to 23 that is adapted for application to a subject or object in need thereof.

26. A bioreactor apparatus comprising the immobilized enzyme complex (IEC) of any one of claims 1 to 23 configured for passage of a liquid comprising the substrate through the IEC.

27. The bioreactor apparatus of claim 26 configured for continuous flow of said liquid through the IEC.

28. The bioreactor of claim 27 configured for recirculation of the liquid through the IEC.

29. A method of enzymatic conversion of a substrate to a desired product comprising the step of exposing the substrate to the immobilized enzyme complex of any one of claims 1 to 25 under conditions where the substrate is converted to the desired product by exposure to the immobilized enzyme complex.

30. The method of claim 29, further comprising the step of recovering the product.

31. The method of claim 30, further comprising; (i) removing the non-covalently bound fusion proteins from the matrix following conversion of substrate to a desired product; and (ii) binding fusion proteins to the matrix.

32. The method of claim 29, wherein the substrate comprises cellulose and wherein the enzyme domains of at least one fusion proteins is selected from the group consisting of a β-glucosidase, an endoglucanase, and an exoglucanase domain.

33. The method of claim 29, wherein the substrate comprises whole blood or red blood cells and wherein the enzyme domain of at least one fusion protein is selected from the group consisting of an α-N-acetylgalactosaminidase, a-galactosidase, or a combination thereof

34. The method of claim 29, wherein the enzyme domain: (i) reduces RDX (hexahydro-1,3,5- trinitro-1,3,5-triazine); (ii) reduces 2,4,6-trinitrotoluene (TNT); (iii) reduces chromium 6+ to chromium 3+; (iv) has 2,2′,3-trihydroxybiphenyl dioxygenase activity; or (v) has enzymatic activity of SEQ NO: 27, SEQ NO: 28, SEQ NO: 29, SEQ NO: 30, SEQ NO: 31, SEQ NO: 32, or SEQ NO: 33.

35. The method of claim 34, wherein the IEC is contained in an enclosure that is permeable to a substrate and product of the enzyme domain of (i), (ii), (iii), (iv), or (v) and comprises the enzyme domain of (i), (ii), (iii), (iv), or (v), respectively.

36. A method of making an immobilized enzyme complex, comprising
(a) covalently attaching biotin or an analog thereof that further comprises a linker molecule to a heat stable matrix selected from the group consisting of a carbon fiber, polylactic acid, polyurethane, polystyrene, silica, nylon, and polypropylene by reacting said matrix with polyethylene glycol (PEG) and polyethyleneimine (PEI) at a ratio of 1 part PEG to 1.25 parts PEI to 1 part PEG to 3.5 parts PEI by weight and reacting the PEG/PEI-treated matrix with an N-hydroxy-succinimide ester of biotin or a biotin analog to obtain a functionalized matrix;
(b) removing any unreacted PEI, PEG, and esters of biotin or the biotin analog from said functionalized matrix; and,
(c) non-covalently attaching at least one fusion protein comprising an enzyme domain and a biotin binding domain (BBD) to a biotin or biotin analog that is covalently attached to the functionalized matrix via a linker molecule.

37. The method of claim 36, further comprising; (i) removing the non-covalently bound fusion proteins from the matrix following conversion of substrate to a desired product by the attached fusion protein; and (ii) binding a fusion protein to the matrix.

38. The method of claim 36, wherein the enzyme domain of at least one fusion protein is selected from the group consisting of an α-N-acetylgalactosaminidase, or α-galactosidase, or any combination thereof.

39. The method of claim 36, wherein the enzyme is selected from the group consisting of a hydrolase, ketoreductase, transaminase, amine oxidase, mono-oxygenase, and an acyl transferase.

40. The method of claim 39, wherein ketoreductase, transaminase, amine oxidase, mono-oxygenase, or acyl transferase domain has a telaprevir precursor compound, sitagliptin precursor compound, or simvastatin precursor compound as a substrate.

41. The method of claim 36, wherein said biotin analog comprises desthiobiotin, 2′-iminobiotin, biotin sulfone, bisnorbiotin, tetranorbiotin, oxybiotin, any derivative thereof, or any derivative of biotin that can be bound by the BBD.

42. The method of claim 36, wherein said linker molecule comprises at least one C2 to C6 alkyl group and at least one amide group.

43. The method of claim 36, wherein said ratio of PEG to PEI is 1 part PEG to 1.5 parts PEI to 1 part PEG to 2.5 parts PEI by weight.

44. The method of claim 36, wherein the enzyme domain of at least one fusion protein is selected from the group consisting of a beta-glucosidase, an endoglucanase, and an exoglucanase domain.

45. The method of claim 39, wherein the hydrolase is a glycoside hydrolase selected from the group consisting of an α-N-acetylgalactosaminidase, α-galactosidase, β-glucosidase, a cellulase, an endoglucanase, and an exoglucanase.

46. The method of claim 36, wherein the enzyme domain has proteolytic activity.

47. The method of claim 46, wherein the enzyme domain with proteolytic activity is collagenase activity.

48. The method of claim 36, wherein the enzyme domain: (i) reduces RDX (hexahydro-1,3,5-trinitro-1,3,5-triazine); (ii) reduces 2,4,6-trinitrotoluene (TNT); (iii) reduces chromium 6+ to chromium 3+; (iv) has 2,2′,3-trihydroxybiphenyl dioxygenase activity; or (v) degrades atrazine and comprises an enzyme domain of SEQ NO: 27, SEQ NO: 28, SEQ NO: 29, SEQ NO: 30, SEQ NO: 31, SEQ NO: 32, or SEQ NO: 33.

49. The method of claim 36, wherein at least two fusion proteins are immobilized on the matrix.

50. The method of claim 49, wherein at least two fusion proteins comprise an enzyme domain that are each independently selected from the group consisting of a beta-glucosidase, an endoglucanase, and an exoglucanase.

51. The method of claim 36, wherein at least one enzyme domain comprises a polypeptide having at least 70% sequence identity to a beta-glucosidase of SEQ ID NO: 2, an endoglucanase of SEQ ID NO: 3, an alpha N-acetylgalactosaminidase of SEQ ID NO: 4, an alpha-galactosidase of SEQ ID NO: 5, SEQ ID NO: 6-33, or SEQ ID NO: 34.

52. The method of claim 36, wherein the BBD comprises an avidin BBD, streptavidin BBD, tamavidin BBD, zebavidin BBD, bradavidin BBD, rhizavidin BBD, shwanavidin BBD, xenavidin BBD, a chimera thereof, or derivative thereof having one or more amino acid residue insertions, deletions, or substitutions.

53. An immobilized enzyme complex made by the methods of any one of claims 36 to 52.

54. The immobilized enzyme complex of claim 53, wherein the IEC comprises a wound healing patch and wherein the enzyme domain enzyme domain has proteolytic activity.

55. The immobilized enzyme complex of any one of claim 1-11, 14, 17-18, or 25, wherein the IEC comprises a wound healing patch and the enzyme domain has proteolytic activity.

56. The method of any one of claims 29-31, wherein the substrate is a wound, and wherein the IEC comprises a wound healing patch, and the enzyme domain has proteolytic activity.

57. A bioreactor, comprising: an immobilized enzyme complex (IEC) that comprises one or more immobilized fusion proteins bound to functionalized, biotinylated carbon fiber matrices to form a heat stable regenerative platform for genetically fused, engineered recombinant enzymes either in a sealed container or in a continuous flow system.

58. The bioreactor of claim 57, further including: an enzyme comprising at least a portion of streptavidin.

59. The bioreactor of claim 57, wherein the biotinylated matrices comprise polypropylene, propylene, or analog thereof.

60. The bioreactor of claim 57, wherein the engineered recombinant enzymes that are expressed by enzyme-encoding open reading frame (ORF) cloned in a Biotin Binding Domain (BBD)-encoding open reading frame (ORF) built-in protein expression vector (pETstra) regulated by a T7 expression system.

61. The bioreactor of claim 60, wherein the engineered recombinant enzymes are configured as streptavidin fused enzymes, antigens, antibodies, or peptides, and that are expressed by a protein expression system and attached to a functionalized surface.

62. The bioreactor of claim 61 wherein the functionalized surface is a biocompatible scaffold.

63. The bioreactor of claim 60 or 61, configured as a continuous flow, multi-enzyme reactor system.

64. The bioreactor of claim 60 or 61, wherein the bioreactor further comprises a biocatalyst device configured to produce one or more therapeutic agents.

65. The bioreactor of claim 64, further comprising IEC that one or more immobilized fusion proteins bound to functionalized, biotinylated carbon fiber matrices to form a heat stable regenerative platform for genetically fused, engineered recombinant enzymes either in a sealed container or in a continuous flow system.

66. A method of using a bioreactor, comprising steps for methods of regeneration of Immobilized Enzyme Complexes following the recirculation of a liquid through an Immobilized Enzyme Complex.

67. The method of claim 66, further comprising steps for: exposing the substrate to the IEC under conditions, recovering a desired product enzymatically converted from a substrate, removing one or more non-covalently bound fusion proteins from a matrix following conversion of the substrate to the desired product, and binding fusion proteins to the matrix.

68. The method of claim 31, further comprising one or more steps for configuring a biofilter to maximize the surface area exposed to genetic engineered recombinant enzymes to form the immobilized enzyme complex wherein the substrate is converted to the desired product.

69. A continuous flow, multi-enzyme bioreactor system, comprising: one or more engineered recombinant enzymes, genetically fused with streptavidin linkers, specific to a regenerated biofilter system having one or more functionalized platforms including a coating selected from a group consisting of carbon, agarose, polystyrene, polypropylene, polyurethane, silica, and nylon.

70. The continuous flow, multi-enzyme bioreactor system of claim 69, wherein the bioreactor system includes one or more Immobilized Enzyme Complexes and the biofilter to form IEC is heat stable

71. An IEC comprising: one or more regenerated functionalized materials, and at least one immobilized enzyme expressed by enzyme-encoding open reading frame (ORF) cloned in a Biotin Binding Domain (BBD)- encoding open reading frame (ORF) built-in protein expression vector (pETstra) regulated by a T7 expression system.

72. The IEC of claim 71 wherein the BBD- fused enzyme is a streptavidin-fused enzyme.

73. The IEC of claim 72 wherein the streptavidin-fused enzyme is selected from the group consisting of endoglucanases, exoglucanases, and β-glucosidase.

74. The IEC of claim 71 wherein the BBD-fused enzymes are immobilized to a biotinylated platform in a ratio of about 1:5.

75. The IEC of claim 74 to biotinylated multiwall carbon fibers.

76. The IEC of claim 73 wherein the streptavidin-fused enzymes are immobilized to a biotinylated platform in a ratio to allow streptavidin-biotin binding to occur in a noncovalent interaction sufficient to eliminate enzyme purification.

77. The IEC of claim 76 wherein the one or more regenerated functionalized materials are associated with a fresh batch of Streptavidin-fused enzymes.

78. The IEC of claim 76 wherein a genetic cassette that is designed for guiding E. coli bacterium in the production of a recombinant enzyme with a genetically fused BBD that is attached to a bio-filter cartridge.

79. The IEC of claim 78 wherein the bio-filter cartridge is configurable to comply flow rate in a corresponding bioreactor system.

80. The IEC of claim 71 is configured to form a multi-enzyme platform to immobilize ketoreductases, transaminases, amine oxidases, mono- oxygenases or acyl transferases.

81. A Bioreactor System, comprising: an enzyme expression system having one or more BBD-fused enzymes immobilized to at least one biotinylated meshed supporting media, a biofilter, that is rapidly regenerated to yield a functionalized polymer platform, wherein the biofilter is immobilized with ionic liquid tolerant cellulases.

82. The Bioreactor System of claim 81, wherein the biofilter further comprises soluble cellulose extracted from biomass feedstock and an ionic liquid pretreatment process hydrolyzed by one or more thermophilic recombinant enzymes tagged with BBDs.

83. The Bioreactor System with the Biofilter immobilized with ionic liquid tolerant cellulases of claim 82, is further configured wherein the one or more thermophilic recombinant enzymes are selected from the group consisting of endoglucanases, exoglucanases, β-glucosidases from Trichoderma reesei, β-glucosidases from Aspergillus spp., thermophilic endoglucanase, Cel5A_Tma form Thermotoga maritima, β-1,4- endoglucanase (Cel5A) from Thermoanaerobacter tengcongensis MB4, endoglucanase and 1,4-β-cellobiosidase from Paenibacillus spp.

84. The Bioreactor System with the Biofilter immobilized with ionic liquid tolerant cellulases of claim 82, is further configured to simultaneously convert free fatty acids and triglyceride into biodiesel, having an enzyme expression system immobilized with one or more lipases to facilitate enzymatic transesterification.

85. The Bioreactor System with the Biofilter immobilized with ionic liquid tolerant cellulases of claim 84, further comprising a biotinylated meshed supporting media and a filter to hydrolyze a soluble cellulose extracted from a biomass feedstock.

86. The Bioreactor System with the Biofilter of claim 85 is further configured as a multi-enzyme system that is immobilized with one or more lipases to facilitate enzymatic transesterification process to simultaneously convert free fatty acids and triglyceride into biodiesel, and wherein the lipases are selected from a group consisting of a Rhizopus oryzae lipase, Candida rugosa lipase, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26.

87. A continuous flow, blood group conversion apparatus, comprising: an IEC with one or more genetically fused, engineered recombinant enzymes, wherein the genetically fused, recombinant enzymes are associated with a protein that specifically binds a functionalized surface of a configurable bio-filter cartridge, and a pump to control flow rates that allow for maximization of blood conversion yields.

88. The apparatus of claim 87, further comprising a platform to deliver antimicrobial proteins, peptides, or antibodies for therapeutic uses wherein one or more recombinant enzymes are immobilized antimicrobial enzymes.

89. The apparatus of claim 88, wherein the immobilized antimicrobial enzymes are selected from the group consisting of lactoferrin, lactoferrin complex, or lysozyme, and wherein the antimicrobial enzymes have antibacterial activity against at least one of Listeria monocytogenes and Clostridium botulinum sub-types.

90. A drug delivery multi-enzyme reactor apparatus, comprising: one or more immobilized fusion proteins including streptavidin, bound to functionalized, biotinylated nanotube material matrices to form a heat stable regenerative platform for producing one or more cycles of genetically fused, engineered recombinant enzymes on a common platform.

91. The continuous flow, drug delivery multi-enzyme reactor apparatus of claim 90 wherein streptavidin fused enzymes, antigens, antibodies, or peptides are expressed by a protein expression system and bound to a functionalized surface selected from a group consisting of carbon multiwall and polypropylene, and wherein the functionalized surface is a biocompatible scaffold.

92. The continuous flow, drug delivery multi-enzyme reactor apparatus of claim 91 wherein the bioreactor further comprises a biocatalyst device configured to form a magnetic a nanobiocatalyst system that is recovered by applying an external magnetic field.

93. The continuous flow, drug delivery multi-enzyme reactor apparatus of claim 92 wherein one or more expressed cellulases are fused with streptavidin and immobilized onto functionalized magnetic carbon-ion nanoparticles.

94. A system for wound healing, comprising: an IEC for conjugation of bioreactive enzymes containing an antimicrobial enzyme, peptide, or enzyme complex on a wound healing patch.

95. The system for wound healing of claim 94, wherein the recombinant enzyme, peptide or complex is genetically fused with a protein that is specifically bound to a functionalized surface of a bio-filter cartridge, wherein the bio-filter cartridge is configured to form an attachable patch.

96. The system for wound healing of claim 95, wherein the recombinant enzyme complex is a glucose oxidase combined with lactoperoxidase (GLG-enzyme complex).


SEQUENCE LISTING (35 sequences)

SEQ ID NO: 1 - protein, 159 aa, Streptomyces avidinii
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 713 aa, Trichoderma reesei
VVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 3 - protein, 417 aa, Trichoderma reesei
NKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 4 - protein, 629 aa, Clostridium perfringens
MKVLGNYIQRNFHYDGKSFYTTSFLNPILNEEILVHTQNEFIIYFVDGEILPSSEMNVEIKKQSEQLLVVNFSKDNLSVEVNYFVENKVINKKLTVFNCCKRINYIDCDTFEFEDTNNIYYPKKQNNIKEMGNFNGYYVELGQPIYAKSLFMGMEFPMGENRIQERKYFSRYYYGKSVEKRLDIHSAIIGAAPEKSKEKIQASFFEYIKAISLPATFRKQYNSWYDHMLNITNDSIIKSFLEINRGFKNYGITLDAFVVDDGWANYESVWEFNDKFPNELKDISECVKNLGSTLGLWIGPRGGYNGTQVTMSDWLEKNKDLNIGSKNKISNDVNVGDFNYLRKMKEKMLEYQSKYDISYWKIDGMLLKPDTEDESGPYGMHTMTAVYEFMISLFNELREERGEKSFWINLTSYVNPSPWFLKWVNSLWIQTSQDVGFTPNGGNDIQKMITYRDSQYYEFLIERDIQLPLCSLYNHEPIYAESASMWYLDHQIYCSIEEFKEYLMFIATRGNAFWEFYYSYSMFDDERWEVNAQAIKWIEENYPILKNSTFFGTKPSLMGVYGYYCQSDSGSKSIISFRNPSDEIKSYKLENIEPKKYDVVLGNKNYKVFEDGSVEVKLNPKEIIILKSK

SEQ ID NO: 5 - protein, 363 aa, Coffea arabica
LANGLGLTPPMGWNSWNHFRCNLDEKLIRETADAMVSKGLAALGYKYINLDDCWAELNRDSQGNLVPKGSTFPSGIKALADYVHSKGLKLGIYSDAGTQTCSKTMPGSLGHEEQDAKTFASWGVDYLKYDNCNNNNISPKERYPIMSKALLNSGRSIFFSLCEWGEEDPATWAKEVGNSWRTTGDIDDSWSSMTSRADMNDKWASYAGPGGWNDPDMLEVGNGGMTTTEYRSHFSIWALAKAPLLIGCDIRSMDGATFQLLSNAEVIAVNQDKLGVQGNKVKTYGDLEVWAGPLSGKRVAVALWNRGSSTATITAYWSDVGLPSTAVVNARDLWAHSTEKSVKGQISAAVDAHDSKMYVLTPQ

SEQ ID NO: 6 - protein, 444 aa, Elizabethkingia meningoseptica
MGALIPSSTLFNIFDFNPKKVRIAFIAVGLRGQTHVENMARRDDVEIVAFADPDPYMVGRAQEILKKNGKKPAKVFGNGNDDYKNMLKDKNIDAVFVSSPWEWHHEHGVAAMKAGKIVGMEVSGAITLEECWDYVKVSEQTGVPLMALENVCYRRDVMAILNMVRKGMFGELVHGTGGYQHDLRPVLFNSGINGKNGDGVEFGEKAFSEAKWRTNHYKNRNGELYPTHGVGPLHTMMDINRGNRLLRLSSFASKARGLHKYIVDKGGESHPNAKVEWKQGDIVTTQIQCHNGETIVLTHDTSLQRPYNLGFKVQGTEGLWEDFGWGEAAQGFIYFEKIMNHSHRWDSSEKWIKEYDHPMWKKHEQKAVGAGHGGMDYFLDNTFVECIKRNEAFPLDVYDLATWYSITPLSEKSIAENGAVQEIPDFTNGKWKNAKNTFAINDDY

SEQ ID NO: 7 - protein, 425 aa, Bacteroides fragilis
MKTPSQTHVLGLAHPPLPMVRLAFIGLGNRGVLTLQRYLQIEGVEIKALCEIREGNLVKAQKILREAGYPQPDGYTGPDGWKRMCERDDIDLVFICTDWLTHTPMAVYSMEHGKHVAIEVPAAMTVEECWKLVDTAEKTRQHCMMLENCCYDPFALTTLNMAQQGVFGEITHVEGAYIHDLRSIYFADESKGGFHNHWGKKYSIEHTGNPYPTHGLGPVCQILNIHRGDRMNYLVSLSSLQAGMTEYARKNFGADSPEARQKYLLGDMNTTLIQTVKGKSIMIQYNVVTPRPYSRLHTVCGTKGFAQKYPVPSIALEPDAGSPLEGKALEEIMERYKHPFTATFGTEAHRRNLPNEMNYVMDCRLIYCLRNGLPLDMDVYDAAEWSCITELSEQSVLNGSIPVEIPDFTRGAWKKCHISRTSDLY

SEQ ID NO: 8 - protein, 459 aa, Shewanella oneidensis
MHNIHRRHFLKAAGAVTAGLVTANIALNANASSVAPKPSSGKSVIGLIAPKMEVVRVGFIGVGERGFSHVEQFCHLEGVELKAICDTHQAVVDRAVEHIVKQKRPKPAVYTGNDLSYRELLNRDDIDIVIISTPWEWHAPMAIDTMESGKHAFVEVPLALTVEECWQIIDTAERTQKNCMMMENVNYGREELMVLNMVRQGLFGELLHGEAAYIHELRWQMKEINHKTGSWRTYWHTKRNGNLYPTHGLGPVSQYMNINRGDRFDYLTSMSSPALGRALYAKREFPADHERNQLKYINGDMSTSLIKTVKGRTIMVQHDTTTPRPYSRHNLIQGTNGVFAGFPNRIAVENDGFGTSYHKWDTDMQKWYDKYDHPLWQRIGKEAEINGGHGGMDFVMLWRMVYCLRNGEALDQDVYDGASWSVVNILSEQSLNNRSNSVNFPDFTRGAWEHAKPLGIVGA

SEQ ID NO: 9 - protein, 468 aa, Tannerella forsythia
MENTRRNFLKKVTAAGIGAAGLAVTDQAMAAVNQPGEAAQQKKKPAGKSDGMLRFGFIGTGSRCQEHINNVLGIQGNKIVAICDIQKGPLEKTLKHIAKFNVPEPKVYTGGEREFEKMLNNEEFDCVIIASPWEWHVPMAVAAMKAGVPYVGVEVSAANTVEECWDLVNVSEATGSHLNILENVCYRRDVMAALRMVREGLFGEMIHGTCGYQHDLRDVKFNDGIHYTYQEGGELRMGPTAYAEAQWRTQHSVTRNGDIYPTHGIGPVANCLNINRGNRFLSLTSMATQSRGLHNFVVDKGGANHPYAKIHFNLGDIVTSMIKCANGQTVIVTHDTNLPRPYSLGFRIQGTRGLWMNDGNHVYVEGQSKPHRWDASDDWFKKYDHKLWSTLELKAKEAGHGGMDYIMMYDFIDAIRNKKPTPMDCYDAAAWSAISGLSEMSIARGGAVVDFPDFTRGQWIHRQPAFAL

SEQ ID NO: 10 - protein, 332 aa, Bacteroides fragilis
MGWSSWNAFRVDISEDIIKHQADLMVEKGLKDVGYHYVNVDDGYFGKRDDNGIMLANEKRFPNGMKPVADHIHSLGMKAGLYTDAGNSTCGSMWDNDTAGIGAGIYGHEPQDAQLYFGDWGFDFIKIDYCGGDALGLNEKERYTSIRNSIDKVNKDASINICRWAFPGTWAKDAATSWRISGDINAHWGSLRYVVGKNLYLSAYAGNGHYNDMDMMVIGFRNDSKVGGQGLTPTEEEAHFGLWCIMSSPLLIGCNLENIPESSLELLKNKELIALNQDPLGLQAYVAQHENEGYVLVKDIEQKRGNVRAVALYNPSDTVCSFSVPFSSLEFG

SEQ ID NO: 11 - protein, 634 aa, Artificial Sequence
  Synthetic metagenomic sequence
MVKRKIFIFMFVLLMSLSLANSNDTKSNLEDMFDYSYLLVPLAVKPSVAGTLRVIEHEGIKTIGDQNGNVIQLRGMSTHGLQWYPEILNDNAFAALSNDWGANVIRLAMYVGEDGYAKDPTVMKERVIKGIELAKKHDMYVIVDWHVHIPGNPLAEIYSGAYDFFDEISNLYPNDPYIIYELCNEPSSNDGGIPGGGVPNNEEGWQIVKSYAEPIIEMLRKKGNENLIIVGSPNWSQRPDLAADDPIADSNTVYAAHFYAGTHKPDPNDYVMSNILYALEKGVPVFISEWGTSEATGAGGPYLEESDKWISFLNKQNISWVNWSLTNKNESSGAFRPFISGQHEAASLDPGEQQIWDPYELSVSGEYVRARIKGISYQPVDRGKSIIWDFNDGTAQGFVVNSDSPIKDLVLQNEKKRLKISGLDSSNDVSEGNFWANARISADNTAVRKDILGCEELKIDVFVTEPTTVAIAAVPQSATHGWANPNCAVKLTEIDFVKQEDGLYKATLSITTEDAPNLGVIATDPNDSTLTNIILFIGTDNADTIFLDNIAIFGEKMVTPVAHAPLGVAKLPSDFEDGTRQGWDWDGTSGVKSALTIENVNNSKALSWEVTYPDVKPGWLGQCPTINPCKYKHN

SEQ ID NO: 12 - protein, 385 aa, Artificial Sequence
  Synthetic metagenomic sequence
MSQFSFTPPCTPAPTGSGWFLLCLLLVFLAGCAGPGAARPSPTVSVPTSAYRATPQGVFRGDEPVPLYGINWFGLETPDRAPHGLWTGRTVADFLAQMRGLGFTAIRLPLSPQVLIPGRPTPSWARSAGYPADAYEGLRYFLGEAQKVGLYVLLDFHTYDPNRIGGKLPGRPFADGYTQADWLADLRRMAELSREFPHIFGVDLCNEPYALTWREWKRLAREGAEAVLSVNPSVLVIVEGVGNASDAGGWPAFWGENLADRDVEIAPERSERILYLPHAYGPSVYRQPYFDAPDFPSNMPAIWDAHFGWMAGKYPLGIGEFGGRYEGDDQVWQDAFADYLLAKGIRIWFYWALNPNSGDTGGVLLDDWETVHRGKMALLRRLMGE

SEQ ID NO: 13 - protein, 910 aa, Paenibacillus odorifer
MIKRYRKQGLVFSLLFTLTFCSNAAFQPAIAEAAAGDYNYAEVLQKSIYFYETQRSGELPDNNRVEWRGDSGLLDGADVGHDLTGGWYDAGDHVKFGLPMAYSTTMLAWSVYEYKQGYEGSGQLEEILDNIRWATDYFVKAHTAPNELWGQVGNGTTDHNWWGPAEVMQMQRPSYKIDATHPGSDLAAETAAALASASIIFRDTDAVYADKLLLHAKQLYNFADTYRGSYSDSITDAKQYYNSWSGYADELSWGAVWLYLATNDQQYLNKAIAASDQWGTNQAGNWGYQWTQSWDDKHYGAQLLLARITGQTKFIQSTERNMQYWTTGVGGTSDRVAYTPGGLAHLDQWGALRYAANQAFMAFVYSDWVSDPVKKDTARSFAERQITYMLGDNPRNSSYVIGYGNNSPQHPHHRTSHGSWNDSQTVPVNHRHVLYGALVGGPSKTDSYTDSINDYVSNEVATDYNAAFTGAIAKMVLLHGQGQQPLAQFPPAETREDEMFVEASVNASGSNFVEIRALLNNRTGWPARASKEMSFNYYVDLSEAIAAGYAPEDITVTAGGYNQGGTVSALQPYDAANHIYYTKVDFTGTLIYPGGQSAHRKEIQFRIAAPLNTNFWNNANDFSFQGVAAQGATPVKTANIPVFDAGVHMYGELPAGGGQPGEPQVPARPTNVQAVAGNGTVHLTWNAISGVSEYTVKRSEVSGGPYTVLENVMGTDYMDSGRVNGTTYYYVITATNSVGESLPSIQVSAKPQETTQPTTGNLKVQYRTNDTNASDGQLRPQFRIMNTGTESVALSGLKLRYYFTVDGDKPQQFHCDYAVVGSGNLSGSFVKLNPASTGADYYLEISFGAGAGSVAPGGDSGEIQARTNKTDWTAYNETDDYSYSAVQQTFADWNKVTLYQGETLVWGLEP

SEQ ID NO: 14 - protein, 1064 aa, Paenibacillus odorifer
MRKKVSVFLALTMFVTIFFSNPIKTNAATPEAPAGWRDLLDYQIFSNISDGWAGDSGFGLETENSKLPIDSTAMYNGLPSLLLNAKTPTSPSWYNALITVAGWKAYDFTSYHPNGFLEFNIKGNAGGESFLLGFKDRVFERAAGNEITTTVNINNYVSITTGWTHVKIPLKDLIQVSQGIDVSSIDALSIKNNGFQPLKVWLNDIRVTSPDKEKEYAPIKVNQLGYPVDGVKQALVTGFEDVLTVDAGTPFNVINATNNSTAYSGTLVLTKNYDAIDSGERIFTADFTNLTIPGKYYVSVQGLQNSPKFTISEANEIYEPFLNDVTRYFYYQRTGINITSPYTQNYQRTDFTPDTAVPLMSNPSIKKDVSKGWYDAGDKGKYVNAGAKALSDLFWAYEMMPEKFTDNQFNIPESGNGIPDILDEARWELEWMLKMQDAATGGFYARVTFQDDDNMVDREIIDKDTVSSRTDIKTTADTATAAGVLAHAYLMYQNIDPAFAQSCLDAAIDAWGYLEAHPENIRTPNTGRWPYDVTDDASNRLWAAGSLYRSTGDAEYNDYFLANYTDMGIFFEDTLDFASGWANTWNTGFFSYLKAANPDSGVLSWYSDKFQQWFNDKVSRYNDSPWKSIIKDGNYYWGITMQVADTPMEMIIGTKLLGTFESNRAIINEINNSQLDWILGQNPVGVSFVSGYGDNSVKYPFSIMYRTDGLPGVPKGYLVGGPNKYSNDITVGNQISRFAAKNYTDNFQEWTTNEHTVYWNSGLVFIAAFATGNSTNSTINPTTAAFDKKTANQADIPVTLTLNGNTLTDIKNGTTSLVVDTDYTVSGNTVTLHKSYLAQQPIGTVNLTFQFNAGSSATLSVAVNDSSVINSTISPTTATFDKQTANQTDIPVTLTLNDNTLIGINNGTTALVAGTDYTVSGTTVTIQKAYLAAQPVGTTTLTFNFSTGASANLAVSVVDTSSTGSGSITVQQYNSPVTATSNSLNPRIKLKNTGTTAINLSDVKLHYYYTIDGEKDQNFWCDWSTVGSSNVTGVFVKLPAPLTGADHYLEIGFTSWKFGCWRQH

SEQ ID NO: 15 - protein, 994 aa, Paenibacillus odorifer
MKLSLIKKPVSVMMAAVLFLSLMTGLFNFRPQTAHASTTEQTRFLQLYAQLKDPANGYFSAEGVPYHAVETLMSEAPDYGHMTTSEAYSYWMWLEVLYGYHTGDWTKLESAWDNMEKYIIPINEGDGKEEQPTMSYYNPNSPATYAAEHAQPDQYPSQLGGQYTAGKDPLDAELKATYGNNQTYLMHWLVDVDNWYGFGNLLNPTHTAAYVNTFQRGEQESVWEAVPHPSQDDKTFGKANEGFMSLFTKEANVPSAQWRYTNATDADARAVQAMYWAKELGYNNTVYLNKAKKMGDFLRYGMYDKYFQKVGSAATDGTPDAGTGKDSNQYLLAWYTAWGGGLGATGNWAWRIGASHAHQGYQNVVAAYALSDSDGGLIPSSATAGQDWENSLKRQLEFYTWLQSSEGAIAGGATNSYQGAYKAYPAGTSTFYGMAYDEAPVYHDPPSNNWFGMQAWSVERVAELYYILASNGDTTSENFQMAKQVIENWVDWSKDYAFANKRPVTDAEGYYLNSQGNRILGGDNPQVATVSAPGEFWIPGNVEWTGQPNTWNGFSGSAANSNLKAVTKSPSQDTGVLGSYIKALTFYAAGTKAEHGSYSTLGGEAQVLAKSLLDTAWGYNDGVGIATTESRGDYSRYFTKEVYFPSGWTGTFGQGNTIPGSSTVPSDPAKGGNGVYASYTDIRPNIVNDSKWQYLLDKYNTSFNKVTKTWDNGAPEFTYHRFWSQVDMATAYAEYDRLINSSVPTEPVAPKAPSKPSVVAGNKVVDLSWNNVTGATSYTVKRAATTGGPYSDVAVVTQATYNDASVVNGTDYYYVVSASNVVGESPNSLEVHAKPIDVPIPVQGDLLVKYRTSDTNPGDNQFRPQLQIVNNGDTAVALSDVKLRYYYTIDGDKPQQFNVDYANIGGSNIQGTFVKVDPAKTGADYYLEISFGAGAGSLAPGANTGDIQIRVNKTDWSNYNEVGDYSYDPAKTSYTEWNHVPLYLNGDLAWGLEP

SEQ ID NO: 16 - protein, 1059 aa, Thermotoga maritima
MQVRKRRGLLDVSTAVLVGILAGFLGVVLAASGVLSFGKEASSKGDSSLETVLALSFEGTTEGVVPFGKDVVLTASQDVAADGEYSLKVENRTSPWDGVEIDLTGKVKSGADYLLSFQVYQSSDAPQLFNVVARTEDEKGERYDVILDKVVVSDHWKEILVPFSPTFEGTPAKYSLIIVASKNTNFNFYLDKVQVLAPKESGPKVIYETSFENGVGDWQPRGDVNIEASSEVAHSGKSSLFISNRQKGWQGAQINLKGILKTGKTYAFEAWVYQNSGQDQTIIMTMQRKYSSDASTQYEWIKSATVPSGQWVQLSGTYTIPAGVTVEDLTLYFESQNPTLEFYVDDVKIVDTTSAEIKIEMEPEKEIPALKEVLKDYFKVGVALPSKVFLNPKDIELITKHFNSITAENEMKPESLLAGIENGKLKFRFETADKYIQFVEENGMVIRGHTLVWHNQTPDWFFKDENGNLLSKEAMTERLKEYIHTVVGHFKGKVYAWDVVNEAVDPNQPDGLRRSTWYQIMGPDYIELAFKFAREADPDAKLFYNDYNTFEPRKRDIIYNLVKDLKEKGLIDGIGMQCHISLATDIKQIEEAIKKFSTIPGIEIHITELDMSVYRDSSSNYPEAPRTALIEQAHKMMQLFEIFKKYSNVITNVTFWGLKDDYSWRATRRNDWPLIFDKDHQAKLAYWAIVAPEVLPPLPKESRISEGEAVVVGMMDDSYLMSKPIEILDEEGNVKATIRAVWKDSTIYIYGEVQDKTKKPAEDGVAIFINPNNERTPYLQPDDTYAVLWTNWKTEVNREDVQVKKFVGPGFRRYSFEMSITIPGVEFKKDSYIGFDAAVIDDGKWYSWSDTTNSQKTNTMNYGTLKLEGIMVATAKYGTPVIDGEIDEIWNTTEEIETKAVAMGSLDKNATAKVRVLWDENYLYVLAIVKDPVLNKDNSNPWEQDSVEIFIDENNHKTGYYEDDDAQFRVNYMNEQTFGTGGSPARFKTAVKLIEGGYIVEAAIKWKTIKPTPNTVIGFNIQVNDANEKGQRVGIISWSDPTNNSWRDPSKFGNLRLIK

SEQ ID NO: 17 - protein, 798 aa, Bifidobacterium thermophilum
MVGSASHPVSDRLYGAFLEDINMSVDGGLNANVVNNYSFDGVYLKHHSIRARGTDRWRTQADPLRFWQFHGVSATSYGTQTRGEHGQRIETSCPAPPLHPNSRYVRVTLPAGRHGADIENLGYNGGDKHAGECAIAISQGHRYDFSVYVRPVAGVVTLAVSVVNASGAALTDCVELSCGGPGDADASSASVAAARACDSDDTAHSNGWVRLTCTLSGMASGLGKLHIGLVPGNGHVGDCGYAGDGADAGEQRADGVRADEHHAGRVATTPATVPTAAPSTAQSAAQLAAPAALSDAVIDLDCVSLMDADTWGADDPKWRYGRLRRDLVEAIAALKPAFLRFPGGCITEGVTPGNEYRWKDTVGALYARRQQYNMWAFRMPDGSSYSQSYQIGFYEYFCLCEDLGAKPLPTLFAGMTCQSPYRDPQSIPVDSDYFRDVVVQDYLDLIEFANGDPDASQWARVRRDMGHPAPFGLDMIGIGNENYGEGYMERFDAIAKAIHEHYPSILCVMSAGLFPYPFSMRRAWNHARDIAAGRGEMLLACDPPLAGSRVIVDEHSYHTPEWFASQARRYDRYPRDSAGVMVGEYSANGYLAGRKQDNAHANTWASALGEAAFLTGCERNSDVVRMTSYAPLLARVPGKGWMQNLIEFDARTVMPTLNAEVEQLFATHIGPMAYECELGDVGHTRAGKAAADRLFASATGDESTRFIKLVNTGGDRIDVTLDISFGLRSLGARSRRDSCRLRVVRLCAAPEARNVLGAEGASHRAVERREDTFAMAGPPVRFALGLPAYSVTMVAVTL

SEQ ID NO: 18 - protein, 317 aa, Thermotoga maritima [sequence omitted]

SEQ ID NO: 19 - protein, 392 aa, Rhizopus oryzae
MVSFISISQGVSLCLLVSSMMLGSSAVPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 20 - protein, 445 aa, Oryza sativa
MSIACCLPVVECVYCLACARWACQHCFHTGGYDSETWGLASPNEFEPVPRLCRLILTVYEDDLEHPQWAPPGGYGIEPRWVVHRKTYEHTGGHAPTYLLYVDHHHSDVVLAVRGMNMAKESDYAVLLDNSLGQRRFDGGYVHNGLLKAAEWLFDAECDVLRDLLERNPGYTLTFAGHSLGSGVVAMLALVAVHNRDRLGGVERKRVRCFAMAPARCMSLNLAVRYADVINSVILQDDFLPRTDTPLEDVFKSLVCLPCLLCGRCLIDTCIPESAMLRDPRRLYAPGRLYHIVERKPFSCRCGRYPPVVRTAVPVDGRFEHIVLSCNMISDHAIIWIEREAQRGLDLMLENERTMKPPETQRMDDEIAIERDHDEEQKAALRRAVALGVADVNVPSAYGTFSENLTPEADEASPVLPDSGLRRTVWDEWIARIFEKDESGKMIPRT

SEQ ID NO: 21 - protein, 549 aa, Candida rugosa
MELALALSLIASVAAAPTATLANGDTITGLNAIINEAFLGIPFAEPPVGNLRFKDPVPYRGSLNGQSFTAYGPSCMQQNPEGTYEENLPKVALDLVMQSKVFQAVLSNSEDCLTINVVRPPGTKAGANLPVMLWIFGGGFEIGSPTIFPPAQMVSKSVLMGKPIIHVAVNYRLASFGFLAGPDIKAEGSSNAGLKDQRLGMQWVADNIAGFGGDPSKVTIFGESAGSMSVLCHLLWNGGDNTYKGKPLFRAGIMQSGAMVPSDPVDGTYGTQIYDTLVASTGCSSASNKLACLRGLSTQALLDATNDTPGFLAFSSLRLSYLPRPDGVNITDDFYALVRNGKYAHVPVIIGDQNDEGTIFGLSSLNVTTNAQAREYFKQSFIHASDAEIDTLMTAYPQDITQGSPFDTGVLNALTPQFKRISAVLGDLAFTLARRYFLNYYQGGTKYSFLSKQLSGLPVLGTFHSNDLTFQNDLLGSGSLIYDNAFIAFVNDLDPNKAGLLVNWPTYTSSSQSGNNMMMINALGLYTGKDNFRTAGYDALFANPPSFFV

SEQ ID NO: 22 - protein, 549 aa, Candida rugosa
MELALALSLIASVAAAPTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 23 - protein, 548 aa, Candida rugosa
MKLCLLALGAAVAAAPTATLANGDTITGLNAIVNEKFLGIPFAEPPVGTLRFKPPVPYSASLNGQQFTSYGPSCMQMNPMGSFEDTLPKNARHLVLQSKIFQVVLPNDEDCLTINVIRPPGTRASAGLPVMLWIFGGGFELGGSSLFPGDQMVAKSVLMGKPVIHVSMNYRVASWGFLAGPDIQNEGSGNAGLHDQRLAMQWVADNIAGFGGDPSKVTIYGESAGSMSTFVHLVWNDGDNTYNGKPLFRAAIMQSGCMVPSDPVDGTYGTEIYNQVVASAGCGSASDKLACLRGLSQDTLYQATSDTPGVLAYPSLRLSYLPRPDGTFITDDMYALVRDGKYAHVPVIIGDQNDEGTLFGLSSLNVTTDAQARAYFKQSFIHASDAEIDTLMAAYTSDITQGSPFDTGIFNAITPQFKRISALLGDLAFTLARRYFLNYYQGGTKYSFLSKQLSGLPVLGTFHGNDIIWQDYLVGSGSVIYNNAFIAFANDLDPNKAGLWTNWPTYTSSSQSGNNLMQINGLGLYTGKDNFRPDAYSALFSNPPSFFV

SEQ ID NO: 24 - protein, 549 aa, Candida rugosa
MKLALALSLIASVAAAPTAKLANGDTITGLNAIINEAFLGIPFAEPPVGNLRFKDPVPYSGSLNGQKFTSYGPSCMQQNPEGTFEENLGKTALDLVMQSKVFQAVLPQSEDCLTINVVRPPGTKAGANLPVMLWIFGGGFEIGSPTIFPPAQMVTKSVLMGKPIIHVAVNYRVASWGFLAGDDIKAEGSGNAGLKDQRLGMQWVADNIAGFGGDPSKVTIFGESAGSMSVLCHLIWNDGDNTYKGKPLFRAGIMQSGAMVPSDPVDGTYGNEIYDLFVSSAGCGSASDKLACLRSASSDTLLDATNNTPGFLAYSSLRLSYLPRPDGKNITDDMYKLVRDGKYASVPVIIGDQNDEGTIFGLSSLNVTTNAQARAYFKQSFIHASDAEIDTLMAAYPQDITQGSPFDTGIFNAITPQFKRISAVLGDLAFIHARRYFLNHFQGGTKYSFLSKQLSGLPIMGTFHANDIVWQDYLLGSGSVIYNNAFIAFATDLDPNTAGLLVNWPKYTSSSQSGNNLMMINALGLYTGKDNFRTAGYDALMTNPSSFFV

SEQ ID NO: 25 - protein, 549 aa, Candida rugosa
MKLALVLSLIVSVAAAPTATLANGDTITGLNAIINEAFLGIPFAQPPVGNLRFKPPVPYSASLNGQKFTSYGPSCMQMNPLGNWDSSLPKAAINSLMQSKLFQAVLPNGEDCLTINVVRPSGTKPGANLPVMVWIFGGGFEVGGSSLFPPAQMITASVLMGKPIIHVSMNYRVASWGFLAGPDIKAEGSGNAGLHDQRLGLQWVADNIAGFGGDPSKVTIFGESAGSMSVMCQLLWNDGDNTYNGKPLFRAAIMQSGAMVPSDPVDGPYGTQIYDQVVASAGCGSASDKLACLRSISNDKLFQATSDTPGALAYPSLRLSFLPRPDGTFITDDMFKLVRDGKCANVPVIIGDQNDEGTVFALSSLNVTTDAQARQYFKESFIHASDAEIDTLMAAYPSDITQGSPFDTGIFNAITPQFKRIAAVLGDLAFTLPRRYFLNHFQGGTKYSFLSKQLSGLPVIGTHHANDIVWQDFLVSHSSAVYNNAFIAFANDLDPNKAGLLVNWPKYTSSSQSGNNLLQINALGLYTGKDNFRTAGYDALFTNPSSFFV

SEQ ID NO: 26 - protein, 549 aa, Candida rugosa
MKLALALSLIASVAAAPTATLANGDTITGLNAIINEAFLGIPFAEPPVGNLRFKDPVPYRGSLNGQSFTAYGPSCMQQNPEGTYEENLPKVALDLVMQSKVFQAVLPNSEDCLTINVVRPPGTKAGANLPVMLWIFGGGFEIGSPTIFPPAQMVSKSVLMGKPIIHVAVNYRLASFGFLAGPDIKAEGSSNAGLKDQRLGMQWVADNIAGFGGDPSKVTIFGESAGSMSVLCHLLWNGGDNTYKGKPLFRAGIMQSGAMVPSDPVDGTYGTQIYDTLVASTGCSSASNKLACLRGLSTQALLDATNDTPGFLSYTSLRLSYLPRPDGANITDDMYKLVRDGKYASVPVIIGDQNDEGFLFGLSSLNTTTEADAEAYLRKSFIHATDADITALKAAYPSDVTQGSPFDTGILNALTPQLKRINAVLGDLTFTLSRRYFLNHYTGGPKYSFLSKQLSGLPILGTFHANDIVWQHFLLGSGSVIYNNAFIAFATDLDPNTAGLSVQWPKSTSSSQAGDNLMQISALGLYTGKDNFRTAGYNALFADPSHFFV

SEQ ID NO: 27 - protein, 474 aa, Pseudomonas sp. ADP
MQTLSIQHGTLVTMDQYRRVLGDSWVHVQDGRIVALGVHAESVPPPADRVIDARGKVVLPGFINAHTHVNQILLRGGPSHGRQFYDWLFNVVYPGQKAMRPEDVAVAVRLYCAEAVRSGITTINENADSAIYPGNIEAAMAVYGEVGVRVVYARMFFDRMDGRIQGYVDALKARSPQVELCSIMEETAVAKDRITALSDQYHGTAGGRISVWPAPATTTAVTVEGMRWAQAFARDRAVMWTLHMAESDHDERIHGMSPAEYMECYGLLDERLQVAHCVYFDRKDVRLLHRHNVKVASQVVSNAYLGSGVAPVPEMVERGMAVGIGTDNGNSNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 28 - protein, 481 aa, Pseudomonas sp. ADP
MTTTLYTGFHQLVTGDVAGTVLNGVDILVRDGEIIGLGPDLPRTLAPIGVGQEQGVEVVNCRGLTAYPGLINTHHHFFQAFVRNLAPLDWTQLDVLAWLRKIYPVFALVDEDCIYHSTVVSMAELIKHGCTTAFDHQYNYSRRGGPFLVDRQFDAANLLGLRFHAGRGCITLPMAEGSTIPDAMRESTDTFLADCERLVSRFHDPRPFAMQRVVVAPSSPVIAYPETFVESARLARHLGVSLHTHLGEGETPAMVARFGERSLDWCENRGFVGPDVWLAHGWEFTAADIARLAATGTGVAHCPAPVFLVGAEVTDIPAMAAAGVRVGFGVDGHASNDSSNLAECIRLAYLLQCLKASERQHPVPAPYDFLRMATQGGADCLNRPDLGALAVGRAADFFAVDLNRIEYIGANHDPRSLPAKVGFSGPVDMTVINGKVVWRNGEFPGLDEMELARAADGVFRRVIYGDPLVAALRRGTGVTPC

SEQ ID NO: 29 - protein, 481 aa, Pseudomonas sp. ADP; identical to SEQ ID NO: 28

SEQ ID NO: 30 - protein, 363 aa, Pseudomonas sp. ADP
MYHIDVFRIPCHSPGDTSGLEDLIETGRVAPADIVAVMGKTEGNGCVNDYTREYATAMLAACLGRHLQLPPHEVEKRVAFVMSGGTEGVLSPHHTVFARRPAIDAHRPAGKRLTLGIAFTRDFLPEEIGRHAQITETAGAVKRAMRDAGIASIDDLHFVQVKCPLLTPAKIASARSRGCAPVTTDTYESMGYSRGASALGIALATEEVPSSMLVDESVLNDWSLSSSLASASAGIELEHNVVIAIGMSEQATSELVIAHGVMSDAIDAASVRRTIESLGIRSDDEMDRIVNVFAKAEASPDGVVRGMRHTMLSDSDINSTRHARAVTGAAIASVVGHGMVYVSGGAEHQGPAGGGPFAVIARA

SEQ ID NO: 31 - protein, 457 aa, Pseudomonas sp. ADP
MKTVEIIEGIASGRTSARDVCEEALATIGATDGLINAFTCRTVERARAEADAIDVRRARGEVLPPLAGLPYAVKNLFDIEGVTTLAGSKINRTLPPARADAVLVQRLKAAGAVLLGGLNMDEFAYGFTTENTHYGPTRNPHDTGRIAGGSSGGSGAAIAAGQVPLSLGSDTNGSIRVPASLCGVWGLKPTFGRLSRRGTYPFVHSIDHLGPLADSVEGLALAYDAMQGPDPLDPGCSASRIQPSVPVLSQGIAGLRIGVLGGWFRDNAGPAARAAVDVAALTLGASEVVMWPDAEIGRAAAFVITASEGGCLHLDDLRIRPQDFEPLSVDRFISGVLQPVAWYLRAQRFRRVYRDKVNALFRDWDILIAPATPISAPAIGTEWIEVNGTRHPCRPAMGLLTQPVSFAGCPVVAAPTWPGENDGMPIGVQLIAAPWNESLCLRAGKVLQDTGIARLKC

SEQ ID NO: 32 - protein, 605 aa, Pseudomonas sp. ADP
MNDRAPHPERSGRVTPDHLTDLASYQAAYAAGTDAADVISDLYARIKEDGENPIWISLLPLESALAMLADAQQRKDKGEALPLFGIPFGVKDNIDVAGLPTTAGCTGFARTPRQHAFVVQRLVDAGAIPIGKTNLDQFATGLNGTRTPFGIPRCVFNENYVSGGSSSGSAVAVANGTVPFSLGTDTAGSGRIPAAFNNLVGLKPTKGLFSGSGLVPAARSLDCISVLAHTVDDALAVARVAAGYDADDAFSRKAGAAALTEKSWPRRFNFGVPAAEHRQFFGDAEAEALFNKAVRKLEEMGGTCISFDYTPFRQAAELLYAGPWVAERLAAIESLADEHPEVLHPVVRDIILSAKRMSAVDTFNGIYRLADLVRAAESTWEKIDVMLLPTAPTIYTVEDMLADPVRLNSNLGFYTNFVNLMDLSAIAVPAGFRTNGLPFGVTFIGRAFEDGAIASLGKAFVEHDLAKGNAATAAPPKDTVAIAVVGAHLSDQPLNHQLTESGGKLRATTRTAPGYALYALRDATPAKPGMLRDQNAVGSIEVEIWDLPVAGFGAFVSEIPAPLGIGTITLEDGSHVKGFLCEPHAIETALDITHYGGWRAYLAAQ

SEQ ID NO: 33 - protein, 274 aa, Pseudomonas putida
MSLQDEALKAWQARYGEPANLPAADTVIAQMLQHRSVRAYSDLPVDEQMLSWAIAAAQSASTSSNLQAWSVLAVRDRERLARLARLSGNQRHVEQAPLFLVWLVDWSRLRRLARTLQAPTAGIDYLESYTVGVVDAALAAQNAALAFEAQGLGIVYIGGMRNHPEAMSEELGLPNDTFAVFGMCVGHPDPAQPAEIKPRLAQSVVLHRERYEATEAEAVSVAAYDRRMSDFQHRQQRENRSWSSQAVERVKGADSLSGRHRLRDALNTLGFGLR

SEQ ID NO: 34 - protein, 380 aa, Thermoanaerobacter tengcongensis
MNKWYAFTGVLVIIIIIMYLILKDTSLTFSCYEREKFPHLMGNALVKKPSVAGRLKIIEKDGRKILGDQYGNPIQLRGMSTHGLQWFSEIINHNAFSGLSRDWESNVIRLAMYVGEGGYATNPSVKDKVIEGINLAIQNDMYVIVDWHVLNPGDPNADIYSGAKDFFKEIATKYPNDLHIIYELANEPNPTPPGVTNDVEGWKKVKNYAEPIIQMLRDMGNQNIIIVGSPNWSQRPDLAAEEPINDPNVMYSVHFYTGTHKVEEHGKPGYVFGNMAKALEKGVPIFVSEWGTSEASGDGGPYLDEADKWLEFLNANNISWINWSLANKNEVSAAFLTSTNLNPGDGKAWAVNQLSLSGEYVRARIKGIPYKPISREARGK

SEQ ID NO: 35 - DNA, 1160 bp, Thermoanaerobacter tengcongensis
tggagtgagatgagactatgaataaatggtatgcttttactggtgtgcttgtgataatcattataatcatgtatttaattttaaaagatacttctttaacttttagctgttatgagagagaaaaatttccgcatttgatggggaatgctttagtaaaaaaaccttcagtagcgggtaggcttaagattatagaaaaagatggaagaaaaatattgggggaccagtatggcaatccaatacagctaagaggaatgagtacacacggattgcagtggttttcagagattattaatcataatgctttttctggattgtcaagagattgggaatcaaacgtgattagacttgctatgtacgttggtgaaggtggttatgctacaaatccaagtgttaaagataaggttatagaaggaataaatttagcaattcaaaatgatatgtatgtaatagttgactggcatgttttaaatccgggagaccctaatgcggacatatacagtggcgcaaaagatttcttcaaggaaatagctactaaatatccaaatgaccttcatataatttatgaattagcaaatgagcccaatccaacccctccaggagtgacaaatgatgttgaggggtggaaaaaagttaaaaactatgctgaaccgataattcaaatgctgagagatatggggaaccaaaatataattattgtaggttctcctaactggagtcaacgcccggatttagcggcagaggaaccaataaatgatcccaatgtaatgtattctgtacatttttatacaggtactcataaagtagaagaacacggtaagcccggatatgtttttggcaatatggctaaagcattggaaaaaggtgtacctatttttgtaagtgaatggggtaccagtgaagctagtggtgatggaggtccatatttagatgaagcagataaatggcttgaatttttaaatgccaataatataagctggataaattggtctttggcaaataagaatgaagtttcagcagcatttttaacatctactaatttaaatcctggtgatggtaaagcatgggctgtaaatcaattgagtctctcaggagaatatgtaagagcaagaataaaaggaattccctataaacctatttctcgtgaggcaagaggaaagtaa